(19) United States
(12) Patent Application Publication
(10) Pub. No.: US 2007/0213786 A1
(43) Pub. Date: Sep. 13, 2007

(54) Closed-loop state-dependent seizure prevention systems

(76) Inventors: James C. Sackellares, Alachua, FL (US); Jose C. Principe, Gainesville, FL (US); Deng-Shan Shiau, Gainesville, FL (US); Panos M. Pardalos, Gainesville, FL (US); Jeongho Cho, Gainesville, FL (US); Sandeep P. Nair, Gainesville, FL (US)

Correspondence Address: EDWARDS ANGELL PALMER & DODGE LLP, P.O. BOX 55874, BOSTON, MA, 02205, US

(21) Appl. No.: 11/641,292

(22) Filed: Dec. 19, 2006

Related U.S. Application Data
(60) Provisional application No. 60/751,595, filed on Dec. 19, 2005.

(51) Int. Cl. A61N 1/36

(52) U.S. Cl. 607/45

(57) ABSTRACT

The invention provides novel closed-loop neuroprosthetic devices and systems for preventing seizures in which control of the delivery of therapeutic electrical stimulation to a neural structure being monitored is determined by the dynamical electrophysiological state of the neural structure. In certain embodiments, a controller which generates predetermined control input is activated based on an Automated Seizure Warning system. Other embodiments of the systems and methods encompass direct control systems wherein the controller design is based on chaos theory. Yet other versions embody model-based control systems in which controller design is based on a model that represents the relationship between the control input and the dynamical descriptor.

### CROSS-REFERENCE TO RELATED APPLICATIONS

The present application claims priority under 35 U.S.C. 119(e) to U.S. Provisional Application No. 60/751,595, entitled Closed Loop Seizure Control Systems, filed Dec. 19, 2005, the disclosure of which is hereby incorporated by reference in its entirety.

### STATEMENT OF U.S. GOVERNMENT INTEREST

Funding for the present invention was provided in part by the Government of the United States under Grant No.: R01-EB002089 from the National Institutes of Health. Accordingly, the Government of the United States may have certain rights in and to the invention.

### BACKGROUND

Several electrical stimulation protocols have been described for treatment of epilepsy in human subjects as well as in animal models of epilepsy. Existing techniques have been designed to directly modulate neuronal firing or to interfere with the synchronization of neuronal populations. Both subthreshold currents as well as superthreshold currents have been used to inhibit neuronal activity. There have been reports of uniform (Ghai et al., 2000) as well as localized (Warren and Durand, 1998) DC electric fields attenuating the bursting in hippocampal slices, although the former has been found to be highly orientation-dependent. Others have investigated anticonvulsant effects of low frequency periodic stimulation. Jerger and Schiff (1995) reported a reduction in frequency of occurrence of tonic phase seizure episodes in the CA1 regions of hippocampal slices using frequencies of 1.0 and 1.3 Hz. Schiff et al. also employed low-frequency pulsed stimuli, the timing or which was derived from a chaos control algorithm, with the aim of reducing the periodicity of high-potassium activity in the CA3 region. Their results showed that the system could be made more periodic or more chaotic by using a strategy of anti-control. However, it is not known to what extent the neuronal firing of the cells that generate the epileptic events was affected by the stimulus.

Most of the stimulation protocols used in clinical studies, with the exception of vagus nerve stimulation (VNS) have employed frequencies upwards of 50 Hz. VNS uses output currents up to 3 mA, pulse width of 250˜500 msec, and frequencies between 10 and 50 Hz (5 Hz for long term stimulation). High frequency stimulation (>50 Hz) on the other hand, has been used in clinical settings to treat the symptoms of epilepsy for the past several decades. Stimulation targets for epilepsy have included the cerebellum, caudate nucleus, hippocampus, thalamus—including the centromedian, anterior and subthalamic nuclei, the vagus nerve, and the epileptic focus itself. Recent animal studies have begun to shed light on the mechanism of action of high frequency stimulation.

Electrical stimulation of the anterior nucleus of the thalamus has been shown to have an anticonvulsant effect on PTZ-induced seizures in rats (Mirski et al., 1997). Current levels between 300 and 1000 mA at 100 Hz were shown to have an anticonvulsant effect while low frequency stimulation of the same target was not effective in inhibiting seizures. Stimulation of the subthalamic nucleus using a 5 second high frequency (130 Hz) train has been found to interrupt ongoing absence seizures in animal seizure models (Vercueil et al., 1998). The effect of subthalamic nucleus stimulation has been reported to be frequency-dependent (Lado et al., 2003). Frequencies of 130 Hz increased clonic seizure threshold, indicating an anticonvulsant effect while stimulation at 260 Hz did not change the threshold. Stimulation at 800 Hz was found to slightly lower the threshold but the changes were not significant. Trigeminal nerve stimulation (Fanselow et al., 2000) at frequencies greater than 50 Hz has been found to reduce PTZ-induced seizure activity by trigeminal nerve stimulation although it is challenging to extend this to the human clinical cases, as the nerve is involved in transmitting both somatosensory and pain information from the head.

The caudate nucleus is another structure that has been explored as a target for stimulation for epilepsy. The effects of stimulation of the caudate nucleus were found to be frequency dependent (Oakley et al., 1982). Stimulation at 0-100 Hz was inhibitory while 100 Hz stimulation increased seizure frequency. Low frequency conditioning stimulation of the epileptic focus (direct stimulation) has been found to suppress kindling caused by 60 Hz stimulation, afterdischarge duration and also seizure intensity. Goodman et al. showed that preemptive delivery of low frequency stimulation decreases the incidence of kindled afterdischarges significantly (Goodman et al. 2005).

Clinical studies in patients with epilepsy have shown an anti-epileptic effect of cerebellar stimulation. Controlled (Krauss and Fisher, 1993) and uncontrolled (Davis et al., 1992) studies have reported improvement in a subset of patients with the former, citing a positive result in a high percentage of cases. Velasco and colleagues reported improvement in seizure frequency and EEG spiking after bilateral stimulation of the centromedian nucleus. Typical stimulation parameters ranged from 60-130 Hz, 2.5-5.0 V and 0.2-1.0 ms duration (Velasco et al., 1987, 2000a, 2000b, 2001). Bilateral anterior thalamic stimulation in five patients with various seizure types was found to cause a significant reduction in seizure frequency, with a mean reduction of almost 54% (Hodaie et al., 2002). The observed benefits, however, did not differ between stimulation-on and stimulation-off periods, suggesting the presence of a placebo effect.

In other studies, bilateral high frequency stimulation of the anterior nucleus produced no observable changes in EEG background or in the interictal spike frequency (Kerrigan et al, 2004). Studies by Velasco (Velasco et al., 2000c) with hippocampal stimulation have revealed the inhibitory nature of subacute continuous hippocampal stimulation. Continuous high frequency stimulation (130 Hz), low-intensity (200-400 mA) stimulation of the hippocampus produced complete blockage of clinical seizures (both complex partial or associated to generalized tonic-clonic) and also significant reduction in epileptiform activity at the epileptic focus. However, the authors stated that appropriate interpretation of results would require studies of extracellular and intracellular recordings in humans. Similar studies have been conducted using amygdalohippocampal stimulation with comparable results (Vonck et al., 2002).

Contingent or closed-loop stimulation techniques may be well suited to overcome some of the limitations of current therapies. Automated seizure detection modalities are currently in place and are being tested in clinical settings. High frequency amygdalohippocampal and anterior thalamic stimulation in patients with mesial temporal lobe epilepsy, triggered by a seizure detection system, has been found to have some beneficial results (Osorio et al., 2005). In that case the evaluation of trials was based only on seizure intensity.

State-of-the-art procedures currently in clinical use are directed only to aborting seizures, and are not capable of preventing their re-occurrence in a subject prone to seizures. A more desirable system for management of patients susceptible to seizure would be fully automated, and would be capable of not only detecting a seizure, but of predicting and preventing the occurrence of an oncoming seizure as well. Such a desirable system would be capable of increasing the time between seizures and ideally eliminating them altogether, without interfering with the cognitive state of the subject.

To achieve such a desirable advance in the field, a critical feature of any automated seizure prediction/prevention system that must be implemented is a control mechanism by which the system determines when and where to deliver an electrical stimulus to the brain of the epileptic subject in need. Existing seizure intervention systems are controlled by what can be termed “naïve” control methodology, meaning that these systems are either limited to measuring the results of the electrical stimulation (such as seizure severity and occurrence), or, when in closed-loop, are triggered only by a seizure occurrence itself. In certain experimental methods under investigation using in vitro brain slices, more sophisticated control concepts involving chaos theory have been used. However, the feasibility of translating such experiments into in vivo control devices remains uncertain.

From the foregoing, it is apparent that there is a clear unmet need for improved control systems in order to achieve the development of fully automated closed-loop systems for seizure intervention and prevention.

### SUMMARY OF THE INVENTION

The invention addresses some of the deficiencies in the art by providing novel state-dependent closed-loop neuroprosthetic devices and related methods for seizure prevention. Control of delivery of therapeutic electrical stimulation in the devices is coupled to a seizure warning/prediction algorithm or other forms of state detection, or in other embodiments involves direct feedback or model-based control schemes. More particularly, in the systems of the present invention, operation (i.e., control of the delivery of therapeutic electrical stimuli aimed at interrupting, delaying, or preventing the occurrence of an impending seizure) is dependent upon the state of a neural structure being monitored that is subject to seizure. Thus, control of the stimulus intervention system is “state-dependent,” i.e., it is dependent upon status information fed back to the device from the neural structure.

This sophisticated level of control is achieved by detecting abnormal seizure-related electrophysiological characteristics of brain activity during the preictal state of an epileptic seizure. Successful intervention, provided in the form of appropriate electrical stimulation, relies on accurate detection of particular dynamical electrophysiological patterns of brain wave activity as determined from electroencephalographic (EEG) recording signals that exhibit identifiable changes during the pre-seizure (preictal) state. Because particular seizure-associated patterns of brain waves are registered in the controller system in advance of an impending seizure, the system is capable of predicting a seizure, and intervening in advance of its progression from the preictal state to the state of seizure (ictus).

Overviews of exemplary closed-loop seizure control systems in accordance with the invention are presented schematically in FIGS. 1 and 2, which are discussed more fully infra. In the closed-loop control systems of the invention, particular seizure-associated features of the brain waves, as detected in electrophysiological recordings, are characterized using algorithms and/or computer simulation models, and the processed information is used to provide input to a controller that interfaces with an electrical stimulator. The stimulator is used to deliver an appropriate electrical stimulus to affected areas of the epileptic brain from which the abnormal brain wave patterns arise.

Typically, the inventive systems are in electrical communication with multiple electrical leads implanted in areas of the brain associated with seizure. Based on feedback from the electrodes, information is processed by the processor and/or controller and electrical stimulation is delivered in a precisely tailored fashion to selected electrodes reporting abnormal brain wave patterns from brain areas experiencing a preictal state, thereby avoiding delivery of unneeded electrical stimulation to brain areas that remain in a normal state.

Accordingly, and in one aspect, the invention provides a closed-loop state-dependent neuroprosthetic device for seizure prevention in which control of the delivery of the electrical stimulus is determined by the dynamical electrophysiological state of a neural structure being monitored. The device includes a detection system that detects and collects electrophysiological information detectable by electroencephalography (EEG) from a neural structure in a subject. Also included in the neuroprosthetic device is an analysis system that evaluates the detected and collected electrophysiological information obtained by EEG, and in real-time extracts from this information electrophysiological features associated with a pre-seizure state in one or more monitored neural structures in the subject.

From the nature of the extracted features, the analysis system determines when electrical stimulus intervention is required. Also included in the device is an electrical stimulation intervention system that provides electrical stimulation output signals having desired stimulation parameters (e.g., duration, pulse width, frequency and intensity) to a neural structure being monitored and in which abnormal neuronal activity is detected.

Further, the analysis system can analyze collected electrophysiological information following electrical stimulation intervention, to assess the short-term effects of the stimulation intervention therapy and to provide feedback to maintain or modify such stimulation intervention.

In some embodiments, the closed-loop neuroprosthetic device of the invention further includes an electrode array suitable for implantation in or on a subject's head, configured to selectively detect electrophysiological information detectable by electroencephalography (EEG), and to output electrical stimulation. Typically, the electrode array is configured so as to create a plurality of channels. Electrical stimulation output signals having desired stimulation parameters (e.g., duration, pulse width, frequency and intensity) can be directed to one or more of the plurality of channels, in which it is predicted or determined that there is the onset of an epileptic state.

Certain embodiments of the closed-loop neuroprosthetic devices comprise an algorithm for automated seizure warning (ASWA). The ASWA can include algorithms for a performing variety of functions, including but not limited to programs for dynamical analysis of EEG signals, for selection of particular electrode groups registering a seizure-associated state for further monitoring and statistical pattern recognition, and for delivery of therapeutic stimulation.

In another aspect, the invention provides a method for preventing or delaying a seizure. The method includes monitoring electroencephalographic (EEG) recording signals in at least one neural structure in a subject fitted with a neuroprosthetic device for seizure prevention wherein control is determined by the dynamical electrophysiological state of a neural structure being monitored. Electrophysiological information obtained from the neural structure is detected and collected and the electrophysiological information is analyzed. A real-time extraction of the collected information is performed, to obtain electrophysiological features associated with a pre-seizure state in a neural structure being monitored. From the real-time extraction of these features, the onset of an epileptic state in the neural structure can be predicted. Appropriate electrical stimulation intervention output signals having desired stimulation parameters (e.g., duration, pulse width, frequency and intensity) are then directed to at least a portion of a neural structure predicted to assume an epileptic state, sufficient to prevent or delay the occurrence of a seizure in the neural structure being monitored.

The method can further include providing an electrode array that is configured to selectively detect electrophysiological information by electroencephalography (EEG), and when needed, to output electrical stimulation signals to the area of the brain being monitored. Typically, the electrode array is configured so as to create a plurality of channels. Providing electrical stimulation output signals can include providing electrical stimulation of desired stimulation parameters (e.g., duration, pulse width, frequency and intensity) to one or more of the plurality of channels, to thereby deliver the stimulation therapy to the brain site in which it is predicted or determined that there is the onset of an epileptic state.

Certain closed-loop feedback embodiments of the method further include collecting electrophysiological information during or following the delivery of electrical stimulation output signals, and analyzing the collected information to assess the short-term effects of the stimulation output signals on the onset of the epileptic state, for example to determine if there is increased or decreased seizure-associated activity, or maintenance of the seizure state. Based on the results of this determination, the stimulation output signals being provided are either maintained or modified.

The neuroprosthetic device and methods of the invention can be used to record and monitor dynamical electrophysiological information from neural structures within any region of the brain known or suspected to be associated with seizure-related activity, including but not limited to the limbic system, hippocampus, entorhinal cortex, CA1, CA2, CA3, dentate gyrus, hippocampal commissure, thalamic nuclei (e.g., anterior and centromedian), subthalamic nucleus, and other basal ganglia.

An especially advantageous aspect of the invention relates to its means for controlling automated operation of the device, in particular with regard to determining when and where to deliver a therapeutic electrical stimulus to prevent or delay a susceptible area of the brain from transitioning from a preictal state to a seizure. The devices and incorporated methods of the invention address this issue in several ways. In some embodiments, control of the parameters (e.g., duration, pulse width, frequency and intensity) of the electrical stimulation intervention output signals is determined by a direct control method in which a control law is derived from the state of a neural structure being monitored by the device. The direct control method can include delay feedback control method or an Ott, Grebogy and York (OGY) method.

In other versions of the invention, control of the parameters of the electrical stimulation intervention output signals (e.g., duration, pulse width, frequency and intensity) is determined by a macroscopic model that utilizes the descriptors of EEG dynamics that describe spatio-temporal patterns in the brain.

Typically, the dynamical descriptors quantify aspects of local signal characteristics associated with seizure activity that is detected in a neural structure being monitored. Several useful dynamical descriptors used in embodiments of the invention involve determining signal dynamics in an electroencephalogram (EEG) over a segment of time, for example utilizing a Short-Term Maximum Lyapunov (STLmax) exponent-based methodology or variations thereof, Kolmogorov entropy, stationarity index, pattern match statistics, or recurrence time statistics.

In other embodiments, control models in accordance with the invention can include hybrid continuous-discrete control schemes, global nonlinear dynamic modeling, or multiple switching local linear modeling.

In various algorithms included in the invention, interdependency between EEG signals can be estimated in several ways, including by use of a T-index, or by direct estimation from a pair of EEG signals.

In some embodiments, the interdependency measure between signals is estimated by using a self-organizing map-based similarity index (SOM-SI).

In some embodiments, the interdependency among a signal group (i.e., more than two signals) can be measured by calculating ANOVA (Analysis of Variance) F-statistics.

Other aspects and advantages of the invention are discussed below.

### BRIEF DESCRIPTION OF THE DRAWINGS

FIG. 1 is a schematic diagram of a closed-loop system 100 for prevention of epileptic seizures, in accordance with an embodiment of the invention.

FIG. 2 is a schematic diagram showing components of a closed-loop seizure prevention system 200, in accordance with an embodiment of the invention.

FIG. 3 is a flow chart illustrating the steps in a real-time automated seizure warning algorithm termed Adaptive Threshold Seizure Warning Algorithm (ATSWA), in accordance with an embodiment of the invention.

FIG. 4 is a schematic diagram illustrating features of a controller that utilizes a low-pass filter, in accordance with an embodiment of the invention.

FIG. 5 is a chart showing a phase portrait of STLmax (a dynamical descriptor of brain state), in precital, ictal and postictal stages of a grade 5 seizure in a rodent brain.

FIG. 6 is a schematic diagram illustrating an embodiment 600 of a closed-loop seizure prevention system featuring model-based control, in accordance with an embodiment of the invention.

FIG. 7 is a schematic diagram illustrating a SOM-based multiple controller scheme for a closed-loop seizure prevention system 700, in accordance with an embodiment of the invention.

FIGS. 8A and 8B are two graphs showing results stabilizing a saddle steady state (8A) and an unstable fixed point (8B) of Lorenz system using a SOM-based controller scheme, in accordance with an embodiment of the invention.

FIG. 9 is a graph showing a pattern match statistics (PMS) profile before, during, and after a seizure, in accordance with an embodiment of the invention.

FIG. 10 is three graphs illustrating Recurrence Time Statistics (RTS) profiles derived from electroencephalograms (EEGs) of human subjects or rats, recorded during epileptic seizures, in accordance with an embodiment of the invention.

FIG. 11 is a graph showing EEG tracings of a representative limbic seizure event in a rodent model of epilepsy, in accordance with an embodiment of the invention.

FIG. 12 is two drawings showing placement of depth and subdural electrodes for recording EEG activity in brains of human subjects with epilepsy, in accordance with an embodiment of the invention.

FIG. 13 is a photograph showing display of EEG recordings using an Automated Warning System (AWS) to control a seizure, in accordance with an embodiment of the invention.

FIG. 14 is a series of graphs showing changes in EEG morphology associated with electrical stimulation, and corresponding changes in dynamical descriptors of brain state (STLmax and T-index) using an AWS, in accordance with an embodiment of the invention.

FIG. 15 is a diagram illustrating time intervals between seizures with and without electrical stimulation to control seizures, delivered in accordance with an embodiment of the invention.

FIG. 16A is a schematic diagram illustrating a state-dependent seizure prevention system 800, in accordance with an embodiment of the invention.

FIG. 16B is a schematic diagram illustrating a seizure prevention system based on direct control 900, in accordance with an embodiment of the invention.

FIG. 16C is a schematic diagram illustrating a seizure prevention system featuring model-based control 1000, in accordance with an embodiment of the invention.

FIG. 17 is a schematic diagram illustrating a seizure prevention device under direct control 1100, in accordance with an embodiment of the invention.

FIG. 18 is a diagram showing a desirable change in the T-index from an unhealthy state characterizing seizure to a healthy state, in accordance with an embodiment of the invention.

FIG. 19 is a diagram showing a control system for a seizure prevention device 1200 based on Multiple Switching local linear models (MSLLM), in accordance with an embodiment of the invention.

FIG. 20 is an EEG tracing showing a STLmax profile over a period of approximately three hours, during which the subject being recorded experienced two seizures, in accordance with an embodiment of the invention.

FIG. 21A shows STLmax profiles recorded from five selected electrodes over a 140-minute period including a 2-minute seizure, in accordance with an embodiment of the invention.

FIG. 21B shows the average T-index curve and threshold of entrainment from the STLmax profiles shown in FIG. 18A.

### DETAILED DESCRIPTION OF THE INVENTION

Electrical stimulation is an established therapeutic intervention used for treating a variety of clinical problems involving the musculoskeletal, neuromuscular, genitourinary, and integumentary systems. This invention provides novel closed-loop devices, systems and methods for control of epileptic seizures, based on electrical stimulation intervention to prevent abnormal brain dynamics and seizure occurrence in brains of epilepsy patients following their detection by the system. Electrical stimuli are delivered to the appropriate regions of the brain using control systems based on feedback from the affected brain areas.

More specifically, and in one aspect, the invention provides a closed-loop state-dependent neuroprosthetic device for seizure prevention. As used herein, the term “neuroprosthetic device” refers to an artificial device adapted to replace or improve the function of an impaired nervous system. Neuroprosthetic devices in accordance with the invention generally comprise a detection system, an analysis system including a controller, and an electrical stimulation intervention system in a “closed-loop” configuration, meaning that activation of the controller depends on the dynamics of the system (state) that is analyzed and monitored by a signal processor. Once the controller is activated, the control output (e.g., the parameters of electrical stimulation) can either be preset (e.g., trained and optimized), or the control output can be adaptively adjusted, based on the responses of the neural structure to the stimulations (i.e., feedback).

The term “seizure prevention,” as used herein, refers to preventing the occurrence, or ameliorating the symptoms of, an impending seizure.

The therapeutic electrical stimulation intervention in the closed-loop devices of the present invention is controlled by the dynamical electrophysiological state of one or more neural structures being monitored in the subject. As discussed, an important operational aspect of the neuroprosthetic devices of the invention is that the delivery of electrical stimulation is “state-dependent.” By “state-dependent,” as used herein, it is meant that the delivery of a seizure intervention (i.e., electrical stimulation) depends upon the detection of a recognized spatio-temporal dynamical state (or pattern) of the system. For example, an electrical stimulator can be activated when an electroencephalographic (EEG) signal processor detects a “dynamical entrainment” (e.g., a gradual decrease in T-index; i.e., slow convergence of STLmax values among a critical/selected EEG channel group). Dynamical entrainment can be defined as a critical value, e.g., a “95% critical value,” at which the T-index curve gradually drops from a value above 5 to below 2.

The detection system detects and collects electrophysiological information that is detectable by electroencephalography from a neural structure in a subject whose brain function is being monitored. As used herein, the term “electroencephalography” (or “EEG”) is used broadly to refer to any known form of recording of electrical activity of the brain, without limitation to the anatomical location in the subject's body for placement of the recording electrodes, or to the type of electrodes used. Another form of EEG involves recording of brain activity using subdural electrodes, to create a record known as an “electrocorticogram” or “ECoG.” In the practice of electrocorticography, an electrode is placed directly on the surface of the brain to record electrical activity from the cerebral cortex. By placing the electrode directly onto the cortical surface, signals from neurons are more effectively recorded than through EEG with electrodes placed on the scalp. One of the limitations of traditional EEG is poor spatial resolution because the skull acts as an attenuator of neural signals, thus filtering out high frequency signals and lowering the signal-to-noise ratio. However, a drawback of ECoG is the requirement of surgery in order to place the electrodes under the dura mater directly onto the brain's surface.

Additionally included within the meaning of electroencephalography, as used herein, is “intracranial EEG” in which brain activity is recorded from electrodes that are placed within the skull, either subdurally (as in ECoG) or intracerebrally, or in both locations.

The analysis system of neuroprosthetic devices in accordance with the invention evaluates the electrophysiological information that is detected and collected by the detection system, and performs a real-time extraction of this information, to obtain electrophysiological features associated with a pre-seizure state in the neural structure. From the extracted features, the analysis system (controller) determines when electrical stimulus intervention is required. The electrical stimulation intervention system provides electrical stimulation output signals having desired stimulation parameters to a neural structure being monitored in which abnormal neuronal activity is detected. The analysis system can further analyze collected electrophysiological information following electrical stimulation intervention, to assess the short-term effects of the stimulation intervention.

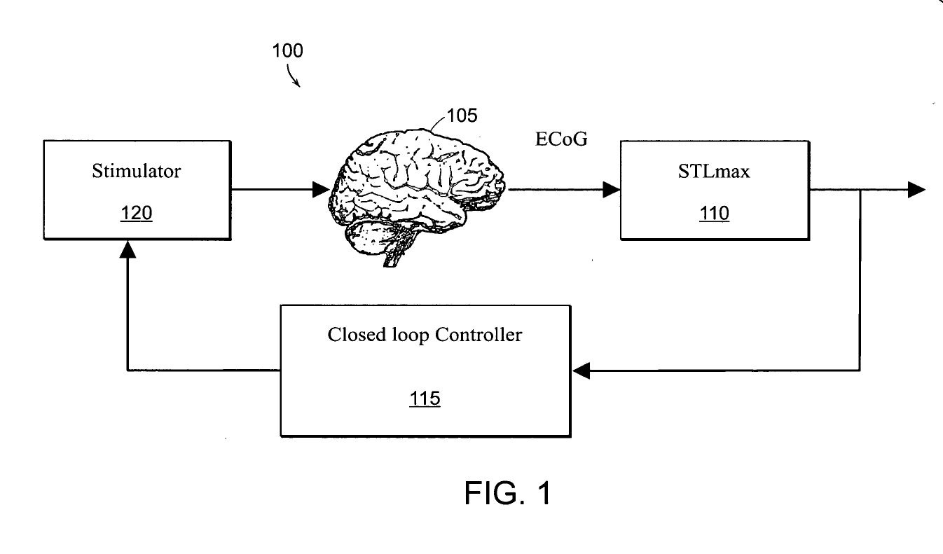

FIG. 1 presents a schematic diagram of a closed-loop state-dependent neuroprosthetic device for seizure prevention 100, in accordance with the present invention. As indicated in the diagram, feedback control of electrical stimulation is dependent upon the state of the brain 105. The state of brain function is detected by the system by electroencephalography, as further described below, in the detector 110. In the particular embodiment illustrated in FIG. 1, the state of the brain is detected in an electorcorticogram but the invention is not so limited.

Device 100 illustrates an embodiment of a seizure prevention system of the invention that is based on the direct control of dynamical descriptors (for example, STLmax), i.e., it is under the direction of a “control law” derived from the state of the system. Based on the control law, controller 115 determines the output of the electrical stimulus delivered by the stimulator 120. In other embodiments of the system, described infra, control is based on the modeling of dynamical descriptors and/or the intervention parameters.

Some embodiments of the closed-loop neuroprosthetic devices interface with automated seizure warning algorithms (ASWA), described in detail infra, that can detect an impending seizure and direct electrical stimulation with appropriate parameters to discrete sites in the brain to increase the time between seizures, and ideally to eliminate seizures, without interfering with the cognitive state of the subject.

The control methods used in the devices and systems of the present invention utilize macroscopic descriptors of the dynamics of the brain. In general, dynamical systems theory is a branch of mathematics describing processes in motion. The dynamical system concept is a mathematical formalization for any fixed “rule” which describes the time-dependence of a point's position in its ambient space. “Dynamical descriptors of brain activity,” as used herein, refer to mathematical measurements that quantify how patterns in EEG recordings of brain activity change over time: “Dynamical descriptors of the preictal state (of seizure)” can include quantifiers of EEG signals, or the spatio-temporal patterns associated with them, that exhibit detectable changes during the preictal state of seizure. For example, certain global dynamical descriptors of brain activity can be used to predict temporal lobe seizures. Dynamical descriptors of the preictal state useful to control the neuroprosthetic devices and systems include, but are not limited to, Short-Term Maximum Lyapanov (STLmax) and variations thereof; Kolmogorov Entropy (K); Stationarity Index (including Pattern Match Statistics, Recurrence Time Statistics); Multidimensional Time Series (including F-statistics, Interdependency Measure Between EEG Signals, Self-Organizing Map-based Similarity Index (SOM-SI)), or the convergence-divergence patterns of any of the above dynamical descriptors.

One useful dynamical measure is the STLmax, which is generated from one or more measures of the signal dynamics that are recorded from a plurality of EEG channels. From this information may be calculated a T-index, which measures the normalized average difference in STLmax values among a group of input EEG channel pairs. When the T-index falls below a given threshold, indicating convergence in the STLmax values, a seizure warning occurs which triggers a controller response. The use of dynamical descriptors such as STLmax and T-index provides spatio-temporal information regarding the state of the neural structure being monitored, and differs from previous methods of monitoring seizure activity using information derived from a single channel to generate a distribution function from an n-dimensional state space representation of EEG signals, broken into segments and compared using “dissimilarity metrics,” such as Chi² statistics and L₁ distance. In prior art systems, when a significant difference is noted in the distribution functions within the sample, a state change is indicated. A single channel method can show that an EEG sample taken at one time is different from that taken at a subsequent time (i.e., can provide temporal information at one space) by creating a state-space representation, and showing that the data from one or more of the segments was from a different state space (therefore a different state). By contrast, dynamical measures as used herein provide spatio-temporal information using integrated input from multiple electrodes. Advantages of the state detection method used in the current invention over prior art methods include: automated selection of critical EEG channels as compared with a subjective selection of a signal channel; spatio-temporal pattern recognition versus temporal-only distribution change detection; and no requirement for pre-set reference window. These advantages provide an automatic and more sensitive system for detecting state changes in multi-channel EEG signals.

As discussed, the control methods used in the present invention utilize macroscopic descriptors of the dynamics of the brain. The macroscopic level of analysis is advantageous because it simplifies the modeling. Based on the reasoning that the epileptic brain when not seizing is stabilized in a temporary “healthy” operating point, it is believed that it may be easier to keep the brain state close to this point than to model the brain dynamics themselves, in absence of presently lacking mathematical knowledge of brain function.

There has been general recognition that electrical stimulation of brain structures involved in the temporal-lobe epilepsy loop can affect the seizure outcome. In contrast to previous ad hoc approaches to combinations of pulse amplitudes and timings for brain stimulation, the present invention incorporates sophisticated methods derived from control theory for exploiting the time dimension, based on measured values of the control variable. Experience from control theory applied to other fields has shown that in complex systems, the control input normally has a narrow dynamic range, and its timing and strength profoundly affect the global dynamics. In complex dynamical systems, the control action must be precisely determined from the present dynamical state and the “error,” which is better achieved with closed-loop control.

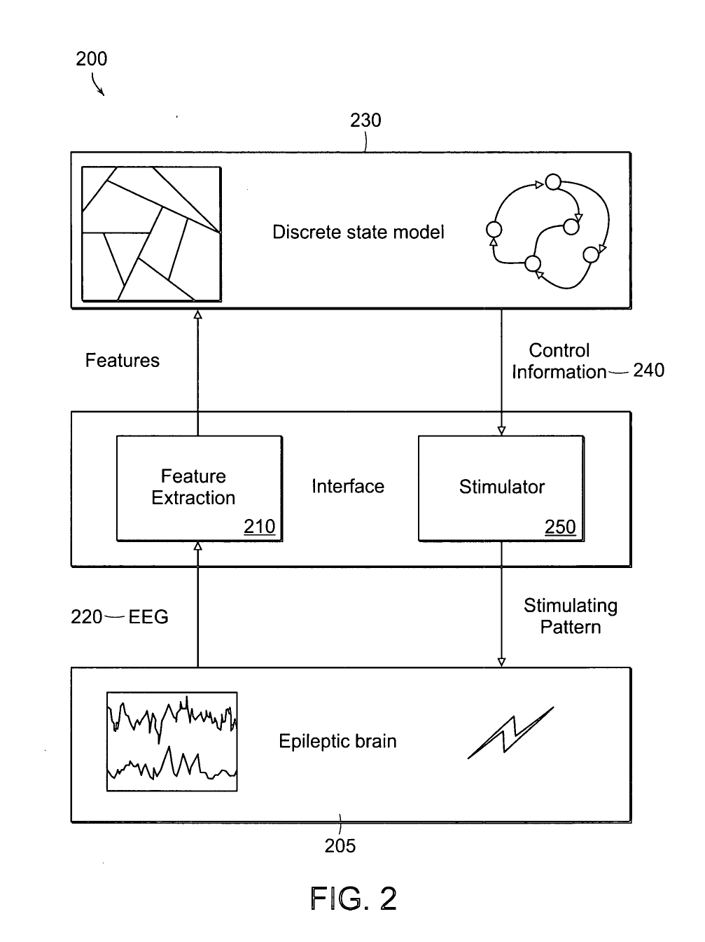

FIG. 2 schematically illustrates the overall control scheme 200 implemented in closed-loop state-dependent seizure prevention systems in accordance with the invention. The control scheme is a hybrid continuous-discrete system because of the physiologic limitations of stimulating brain tissue. Hybrid control is a novel method to control complex systems such as transportation systems, manufacturing processes and chemical processes (Stiver and Antsaklis 1992; Antsaklis 2000; Bemporad 2004; Bemporad and Morari 2001; Koutsoukos et al. 2000). A hybrid system in accordance with the invention comprises two distinct components: a continuous plant and a discrete-event controller.

The brain is in fact a continuous dynamical system, as is its output, the electroencephalogram (EEG). However, stimulation by the neuroprosthetic systems is done at discrete intervals, with the goal of minimizing the use of electrical stimulation. Referring to FIG. 2, dynamical features 210 are extracted from the EEG 220 of the epileptic brain and are processed by different control methodologies as described below, to implicitly or explicitly model the brain states. The output of the model 230 then provides control information 240 as input to the stimulator module 250, in a closed-loop fashion.

### Algorithms for Automated Seizure Warning Systems

In one aspect, the invention provides automated seizure warning algorithms (ASWAs) that interface with the closed-loop seizure control systems. A reliable ASWA is a highly desirable element for seizure control systems. These algorithms comprise several components, including dynamical analysis of electroencephalogram (EEG) signals; optimization algorithms for selection of critical electrode groups; and statistical pattern recognition methods.

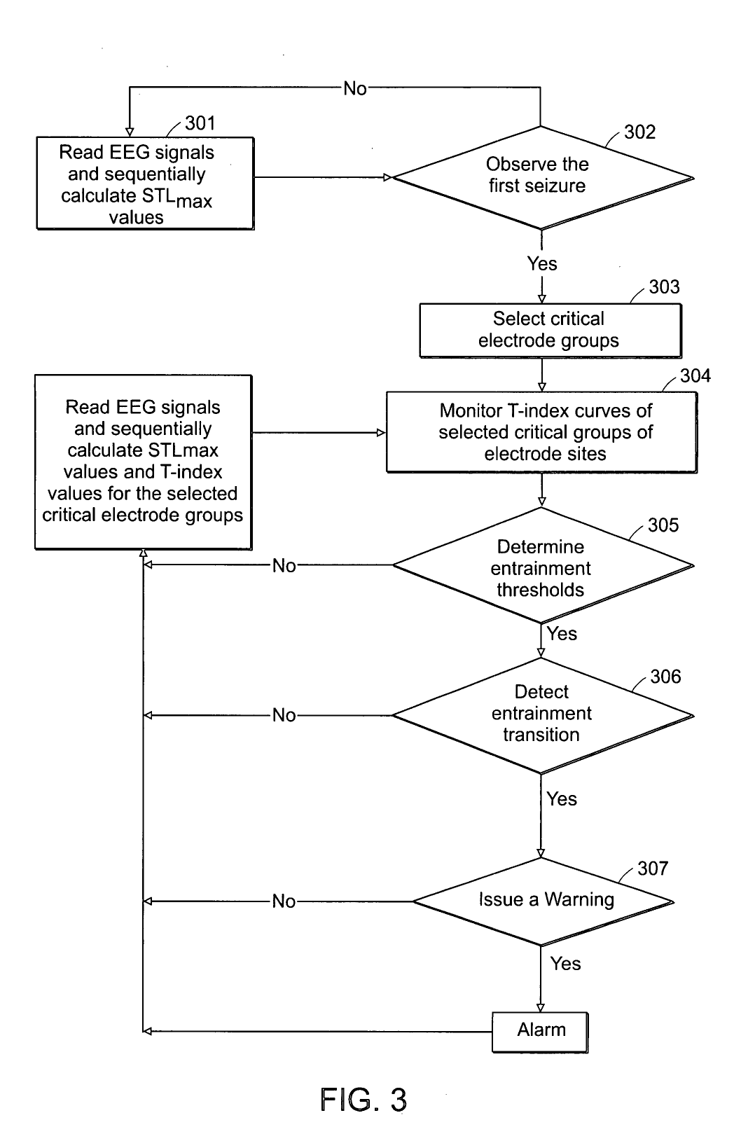

One preferred embodiment of an ASWA in accordance with the present invention is an algorithm termed an “adaptive threshold seizure warning algorithm (ATSWA).” A flow chart of an exemplary ATSWA is shown in FIG. 3. ATSWA can be run in real-time for at least 60 EEG channels on a 2 GHz Pentium personal computer. This algorithm and its use are further described in co-pending applications U.S. patent application Ser. No. 10/648,354, PCT/US2003/026642, filed Aug. 27, 2003, and U.S. patent application Ser. No. 10/673,329, filed Sep. 30, 2003, hereby incorporated by reference in their entireties.

In one embodiment, an ATSWA is based on measurements of STLmax. STLmax describes the signal dynamics of an EEG over a segment of time for each recording channel. It quantifies the observed local chaoticity of a dynamical system, and is closely related to the average rate at which information is produced (Mayer-Kress and Holzfuss, 1986). The basis for the use of STLmax is that the epileptic brain progresses into and out of order-disorder states, according to the theory of phase transitions of nonlinear dynamical systems (Iasemidis and Sackellares, 1996). Details of the method for calculating STLmax from nonstationary signals have been described (Iasemidis et al., 1990; 1991).

The flow chart in FIG. 3 shows the steps in a real-time automated seizure prediction algorithm. STLmax describes the EEG signal dynamics over a segment of time for each recording channel. Referring to Step 301 in FIG. 3, as EEG signals are collected, an estimation of STLmax is performed, for example, for every 10.24-second window, in all channels, creating a new time series of STLmax profiles with a 10.24 sec time resolution. Based on a patient's STLmax time profiles from all recording channels before and after the first available seizure, ATSWA selects one or more critical groups of EEG channels for prospective monitoring. The first seizure in the record is manually indicated by the attending clinician, or detected automatically by a seizure detection algorithm to initiate the algorithm (FIG. 3, Step 302). Once the algorithm is initiated, the ATSWA automatically selects the EEG channels to be employed for prediction of the subsequent seizures (Step 303).

The channel selection is performed automatically, based on a similarity index of STLmax profiles (within 10 minutes before and after the first seizure) called the T-index, derived from the paired T-statistic. The T-index for any given pair, for example calculated over a 10-minute window, is the absolute value of the mean difference in STLmax values divided by the standard deviation. The critical groups of electrode channels are defined as the groups of channels which maximize the quantity, T(postictal)−T(preictal), where T(postictal) is the average T-index in the 10 minute window following the offset of the first seizure, and T(preictal) is from the 10 minute window preceding the first onset. The selection of 10-minute intervals before and after the seizure in this process is advantageous, based on our studies on dynamical resetting of epileptic seizures (Iasemidis et al, 2004).

This task is accomplished by creating two T-index matrices (one before and one after the first recorded seizure). The channels thus selected are named “critical electrode sites.” Groups of critical electrode channels are selected for use in predicting subsequent seizures. Two parameters (number of groups and number of channels per group) are selected based on a prediction sensitivity analysis from a set of seizures in patients (e.g., first four seizures from each patient). The average T-index values of these groups are then monitored forward in time (e.g., moving window of 10.24 seconds at a time), generating T-index curves over time (Step 304).

Referring to Steps 305 and 306 in FIG. 3, an entrainment transition is detected when the average T-index curve for any of the groups falls below a dynamically adapted critical threshold. The adaptive threshold includes a “dead-zone” with an upper threshold (UT) and a lower threshold (LT). The UT is determined as follows: if the current T-index value is greater than max20 (i.e., the maximum T-index value in the past 20 minute interval), UT is set equal to max20; otherwise, the algorithm continues searching sequentially in time to identify UT. Once UT is identified, the lower threshold LT is set equal to UT−D, where D is a user-controlled variable in T-index units. After UT and LT are determined, an entrainment transition is detected if an average T-index curve is initially above UT and then gradually (e.g., within at least 30 minutes of traveling time) drops below LT (FIG. 3, Step 306). Once an entrainment transition is detected, the algorithm returns to Step 305 to search for a new UT to be used for detection of the next transition.

We hereafter use the notation U_{T}^{ij} and L_{T}^{ij} to denote the ith group of channels whose average T-index satisfies the necessary conditions for detection of the jth transition. Those of skill in the art will recognize that the parameters of 20 minutes for determining U_{T} and 30 minutes for traveling time are empirical and that other parameters can be used.

After an entrainment transition is detected, the algorithm determines whether a seizure warning should be issued (FIG. 3, Step 307). In this algorithm, if a transition is detected within the prediction horizon from the previous warning, the transition is considered as part of a cluster of transitions (due to a possible cluster of impending seizures), and in this case a new warning is not issued. Thus, a seizure warning defines mathematically the beginning of a new dynamical EEG state called the “preictal transition.”

### Direct Control Framework Based on Dynamical Descriptors

Some embodiments of the closed-loop neuroprosthetic devices and systems incorporate direct control methods, in which a “control law” is derived from the system state, to provide input to the stimulator module. Various embodiments of the system utilize approaches shown to be useful in the control of complex dynamical systems with high sensitivity to initial conditions, including the Delay Feedback Control (DFC) method and the OGY method.

1. Delay Feedback Control Method.

This is a relatively simple technique that can be applied to a large class of complex dynamical systems that are sensitive to initial conditions, which are commonly called “chaotic systems,” but are not limited to these (Pyragas, 1992). DFC utilizes feedback of the output of a system to its input, combined with a delayed and processed version of the output. One advantage of the technique for the application to epilepsy seizure warning systems is that the system dynamical equations are not necessary, in order to apply the technique. However, a difficulty is the choice of the operating point to be controlled, and the parameterization needed in the delay.

Although the field of controlling chaos deals mainly with the stabilization of unstable periodic orbits, the problem of controlling the system dynamics on unstable fixed points (non-oscillatory solutions) is attractive for epilepsy control using global dynamical descriptors because our previous work showed that the “healthy” operating point of the brain can be translated in relatively constant large values of the STL max (Iasemidis and Sackellares, 1991). Usual methods of classical control theory are based on proportional feedback perturbations, and require knowledge of the location of the unstable fixed point in phase space (Stephanopoulos, 1984), which means the specific value of the STLmax and the equations of motion must be specified. Because these constraints are not realistic, adaptive, reference-free control techniques, capable of automatically locating the unknown steady state, are required.

It can be straightforward to attain adaptive stabilization of the unknown steady state based on derivative control (Bielawski et al. 1993; Parmananda et al. 1994). Indeed, the control perturbation is derived from the derivative of an observable, and therefore the perturbation does not influence the steady-state solutions of the original system, since it vanishes whenever the system approaches the steady state. For fixed-point control, an adaptive controller can be designed on the basis of a finite-dimensional dynamical system.

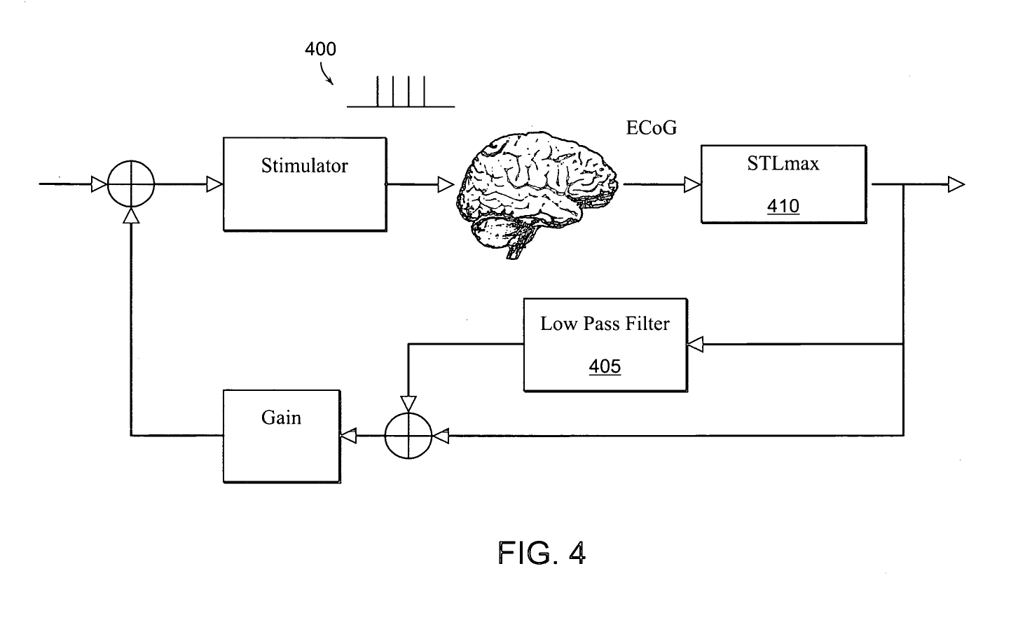

As shown schematically in FIG. 4, an example of such a controller is illustrated in device 400, which utilizes a conventional low-pass filter 405 having only one inherent degree of freedom. The filtered output signal of the system estimates the location of the fixed point, so that the difference between the actual and filtered output signals can be used as a feedback perturbation. Several groups have demonstrated the efficacy of this methodology in synthetic chaotic systems (Namajunas et al. 1995; Rulkov et al. 1994), and in lasers (Ciofini et al. 1999).

The control is exercised on the STLmax 410, or any other dynamical parameter for which the range of values that correspond to “healthy” conditions (e.g., desired STLmax), is known. The method functions according to the following calculations:

An autonomous dynamical system is described by ordinary differential equations \dot{x}=f(x,d) where the vector xεR^{m} defines the dynamical variables and d is a scalar parameter available for an external adjustment, such as the desired STLmax. We envision a scalar variable y(t)=g(x(t)) that is a function of dynamical variables x(t) that can be measured as a system output. Assuming that at d=d₀ the system has an unstable fixed point x* that satisfies f(x*, d₀)=0, if the steady state value y*=g(x*) of the observable corresponding to the fixed point were known, we could stabilize the system by using a standard proportional feedback control, i.e., adjusting the control parameter by the law d=d₀−k(y−y*). However, supposing that the reference value y* is unknown, our aim is to construct a reference-free feedback perturbation that automatically locates and stabilizes the fixed point. Such a perturbation should vanish when the system settles on the fixed point.

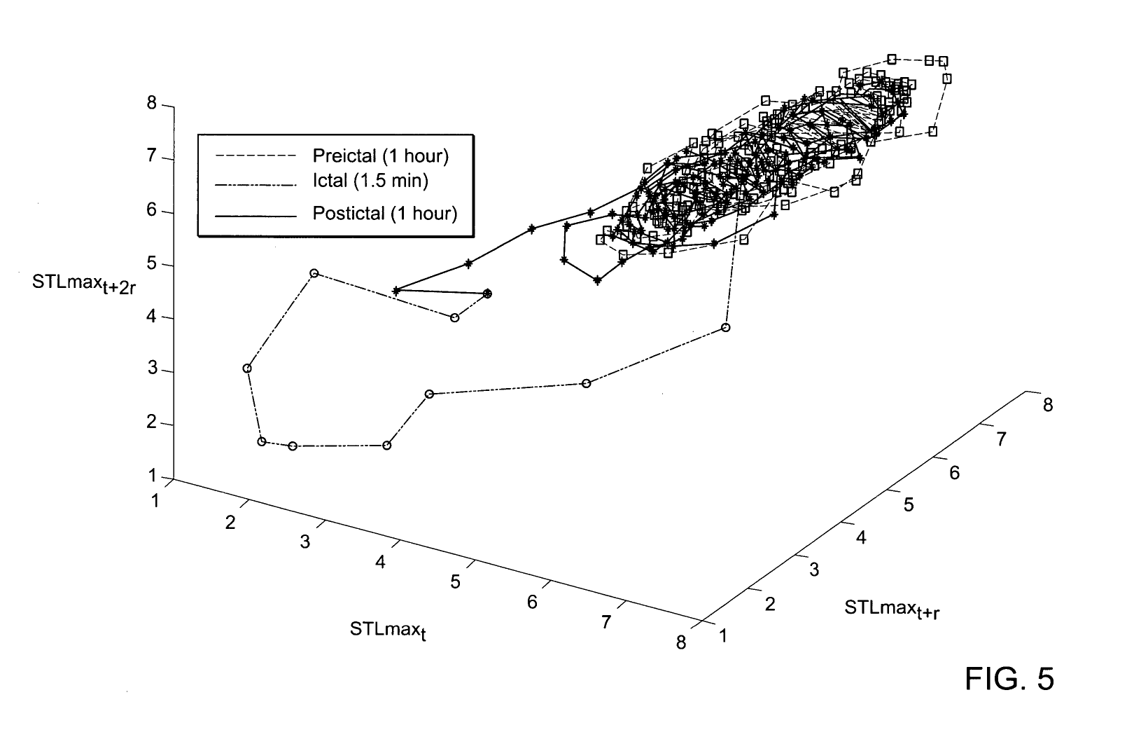

A simple controller satisfying this requirement that can be constructed on the basis of a one-dimensional dynamical system is a low pass filter \dot{w}=w_{c}(y−w) where w_{c} is its cutoff frequency and w is its dynamic variable. The stimulator translates the control input into a set of pulses. A known relation between the stimulator input and the number of pulses is established a priori under experimental conditions. Since the stimulator is within the feedback loop, the controller gain self adjusts, taking into consideration the transfer function of the stimulator. The assumptions underlying this methodology have been experimentally observed in STLmax time series data from human patients and rodents, as shown in Examples, infra. FIG. 5 is a graph from these studies showing a phase portrait of STLmax during a grade 5 seizure in a rodent.

The normal brain seems to work at a relatively constant STLmax (the control parameter), which means that a homeostatic equilibrium point for the healthy brain dynamics may exist, as has been postulated by Haken among other researchers (Haken 1996). It is therefore plausible that the dynamical equation for brain dynamics can be written as a parametric function of the state x(t) and the homeostatic equilibrium d. A practical difficulty is to find d, but since this is a single constant parameter, an efficient Fibonacci search can be conducted to find an appropriate value.

Alternatively, approaches can be used to estimate d. One approach is to use the average STLmax during long periods as the desired response. A second alternative instead of using a determined d is to use a so-called “dead zone controller” wherein the control loop is open until the STLmax falls below a predetermined value (e.g., the value used for the seizure warning alarm). At that point, the value of the T-index is used as d, i.e., the system tries to stabilize the STLmax at that value. The second method has the advantage of avoiding any stimulation when the STLmax is within the normal region.

The parameters of the controller include the low pass filter transfer function and the gain. In some instances, it may be preferable to use a bandpass filter instead of the lowpass filter, if the signal obtained from the subtraction of the STLmax and its lowpassed filtered version (which is a high pass filter) is too noisy. In this condition, the highpass cutoff can be determined experimentally with the available STLmax data, to ensure a smooth signal at all times. Because the model parameters are unknown, both the gain and the lower cutoff of the filter are adaptively determined from the data.

In some embodiments, similar to state-dependent control systems, the operating point of the controller can be chosen based on the ASW algorithm. Once a preictal state is detected by ASW algorithm, the controller will be activated to determine the most appropriate stimulation output (parameters, e.g., stimulation intensity, frequency, duration, etc.). These parameters can be determined automatically by the controller based on the feedback response measure: Dy=yₜ−yₜ*, where yₜ is the T-index value at time t, and yₜ* is the low pass filter value of yₜ. The filtered output yₜ* is used to estimate the location of the fixed point, so that the difference Dy can be used as a feedback perturbation. In addition, another condition can be added such that the controller is activated to avoid “unhealthy” region even though Dy=0. The aim is to construct a reference-free feedback perturbation that automatically locates and stabilizes the T-index values in the fixed point region. The remaining step is to find the optimal relationship (i.e. the “gain”) between Dy and the stimulation parameters that will give the best control performance.

2. OGY Method.

Nonconvergent (chaotic) dynamical systems contain a huge number of unstable periodic orbits. These orbits represent genuine motions of the system and can be stabilized by applying tiny control forces. Hence, chaotic dynamics opens the possibility to use flexible control techniques and stabilize quite distinct types of motion in a single system with small control power. This recognition was the essential contribution of Ott, Grebogy and York, and their innovative methodology (termed “OGY”) has resulted in numerous applications of chaotic control (Ott et al. 1990).

OGY methodology can be used as an alternative method to the DFC method for seizure control. Additionally, the OGY control method is very effective if saddle points exist in the attractor, since the method relies upon the identification of saddle instabilities; i.e., unstable periodic points located on a surface having both stable and unstable directions. For the seizure control application, a local map around the desired attractor is constructed from the observation of STLmax (or equivalent descriptor), to obtain a periodic orbit by the method of delay-coordinate embedding, due to the lack of information about the brain model. The system approaches the periodic point along a stable direction and diverges away from it along an unstable one. When the delay-coordinate vector of STLmax is in the neighborhood of the desired attractor, a perturbation (electrical stimulation) is applied to the brain, such that on the next iteration the STLmax falls on the stable direction, with a corresponding transition from periodic (ictal) to chaotic (interictal) behavior. The state then moves towards the attractor in successive iterations.

The concept of the method is as follows. The controlled system can be described by the state equation, \dot{x}=f(x,u), and the desired trajectory x*(t) can be a solution of \dot{x}=f(x,u) for u=0. This trajectory may be either periodic or chaotic; in both cases it is recurrent. We construct the surface (so-called Poincare section), s={x:s(x)=0}, passing through the given point x₀=x*(0) transversally to the trajectory x*(t) and consider the map x→P(x,u) where P(x,u) is the point of first return to the surface S of the solution of \dot{x}=f(x,u) that begins at the point x and was obtained for the constant input u. Owing to the recurrence of x*(t), this map is defined at least for some neighborhood of the point x₀. By considering a sequence of such maps, we get the discrete system xₖ₊₁=P(xₖ,uₖ).

The next step in designing the control law lies in replacing the original system by the linearized discrete system \tilde{x}ₖ₊₁=A\tilde{x}ₖ+Buₖ, where \tilde{x}ₖ=xₖ−x₀. Stabilizing control is determined for the resulting system as, for example, the linear state feedback uₖ=Cxₖ. The final form of the control law is uₖ={C\tilde{x}ₖ if ∥\tilde{x}ₖ∥≦Δ, 0 otherwise}, where Δ>0 is a sufficiently small parameter. A key point of the method is to apply control only in some vicinity of the goal trajectory by introducing an “outer” dead zone. This has the effect of bounding control action. Using this principle, many physical systems exhibiting chaotic behavior have been subjected to control.

### Model-Based Control Framework Based on Dynamical Descriptors

Model-based control provides the opportunity to explicitly model the dynamics of spatio-temporal parameters, and to experiment with synthetic realistic models of brain dynamics. As used herein, “spatio-temporal electrophysiological state of a neural structure” refers to an electrophysiological state that is characterized, e.g., by the spatio-temporal pattern of EEG signals in the brain or portions thereof being monitored. A spatio-temporal pattern of EEG signals contains information that is extracted and/or analyzed from EEG signals both in space (e.g., across different brain areas) and temporally (over time).

It is well known from control theory that the knowledge of the model makes the controller much easier to build and more accurate and robust. These properties can extend to the design of controllers for nonlinear systems. Indeed, once the Lorenz system was identified by the local linear switching models, a controller was easily derived (just a linear inverse) that put the chaotic system in basically any point in state space, reliably and fast. However, when compared with direct control, model-based control requires an extra step of system identification (Narendra 1990).

Empirical evidence indicating the possible relevance of chaos to brain function was first obtained by Walter J. Freeman, through his work on the large-scale collective behavior of neurons in the perception of olfactory perception (Skarda and Freeman 1987; Freeman, 1975). These findings suggest that a separate chaotic attractor is maintained for each stimulus, and the act of perception consists of a transition of the system from the domain of influence of one attractor to another. In a chaotic attractor, the system state may be, at any given time, infinitesimally close to any one of the infinite periodic attractors, but due to the highly unstable nature of the periodic orbits, the periodicity is never manifested over a measurable period of time. These characteristics have attracted many researchers to the area. For a recent review of many important works in the area of chaotic control, see Andrievskii and Fradkov, 2003.

In 1990, the possibility that chaos can be controlled efficiently using feedback schemes, i.e., by converting the chaotic behavior of a system to a time-periodic one, was described by Ott, Grebogi and Yorke (OGY). A special case of the OGY method was introduced by Hunt in 1991, termed “occasional proportional feedback,” which is used for stabilization of the amplitude of a limit cycle and is based on estimating the local maxima (or minima) of the output. A modification of proportional perturbation feedback (PPF) called stable manifold placement (SMP), which is simpler and more robust than PPF has also been described (Christini et al., 1997). This method requires fewer assumptions to be made about the system parameters and has been used in modification of bursting behavior in hippocampal slices (Slutzky et al., 2003). However, the control application is dependent upon the dynamics (since the perturbation is synchronized with the intrinsic fly by around the unstable point). It also requires knowledge about the system, which most often is unavailable.

An alternative is to modify the chaotic dynamics themselves into periodic orbits. In recent years there has been increasing interest in the method of time-delayed feedback (Pyragas, 1992) in which the control input is fed by the difference between the current state and the delayed state. The delay time is determined as the period of the unstable periodic orbit to be stabilized. Hence, the control input vanishes when the unstable periodic orbit is stabilized. In addition, this method requires no preliminary calculation of the unstable periodic orbit. Hence, it is simple and convenient for controlling chaos. Reported applications of this method include stabilization of coherent modes of lasers (Bleich et al., 1997; Loiko et al., 1997), control of cardiac conduction model (Brandt et al., 1997), and paced excitable oscillator described by the Fitzhugh-Nagumo model widely used in physiology (Bleich and Socolar, 2000). In addition, a robust local controller, the decentralized delayed feedback control, has been proposed in (Konishi, et al., 2000) showing some advantages relative to the conventional delayed feedback control. As another robust controller, a simple feedback control design method was proposed (Jiang et al., 2004) by using some ideas from the state observer approach. Especially, this method was demonstrated to be highly robust against system parametric variations in system parameters.

Alternatives to analytical control algorithms considered to control chaos involve some intelligent control techniques, e.g., neural networks (Hirasawa et al., 2000a; Hirasawa et al., 2000b; Poznyak et al., 1999), fuzzy control (Chen et al., 1999; Tang et al., 1999), etc. Specifically, neuro-genetic controllers for chaotic systems were proposed in (Dracopoulos and Jones, 1997) using large control adjustments and in (Weeks and Burgess, 1997) using small perturbations without a priori knowledge of the dynamics, even in the presence of significant noise. Recently, the cerebellar model articulation controller (CMAC) has been adopted in (Lin et al., 2004) for the control of unknown chaotic systems, and the weights of the CMAC were online tuned by an adaptive law based on the Lyapunov sense.

Another control possibility to be considered is multiple model-based control that we have proposed (Cho et al., 2004) in which we have attempted to control unknown chaotic systems using data-driven multiple models. The concept of multiple models with switching has been employed in order to simplify both the modeling and the controller design. Multiple models are plausible if a function f to be modeled is complicated because global nonlinear models may not be capable of approximating f equally well across all space. In this case, the dependence on representation can be reduced using local approximation where the domain of f is divided into local regions and a separate model is used for each region. Local linear models are chosen and derived through competition using the Self-Organizing Maps (SOM) (Kohonen, 1995).

The two primary methodologies to implement system identification are (1) the input output model or (2) prediction. For dynamic modeling (i.e., system identification of chaotic time series), the primary method is iterative prediction as explained by Haykin and Principe 1998. The results of this method give very exciting results for real signals (laser instability and sea clutter, for example), but they have not been applied to EEG. The high dimensionality of the system (brain) is an enormous difficulty, as is the eventual time varying system parameters. For this reason in one aspect the invention models the dynamics of the STLmax, due to the intrinsic simplification that occurs when order parameters are used (Haken 1996).

To implement the system identification methodology, the invention utilizes two types of models that have been applied successfully in nonlinear control engineering: global nonlinear modeling and multiple switching local linear models.

1. Global Nonlinear Dynamic Modeling.

Recurrent neural networks (RNN) and time lagged feedforward neural networks (TLFNs) are capable of learning and reproducing chaotic dynamics in a variety of realistic and synthetic nonlinear systems. Haykin and Principe (1998) used TLFNs (delay line followed by a radial basis function network or multilayer perceptron) trained in an iterative prediction configuration using backpropagation through time (Werbos, 1990). This basic methodology for dynamic modeling is used with global models because it is well established and gives good results (Elman, 1990). Recurrent neural networks are even more powerful (Siegelmann, 1993), but the issue is the difficulty in training.

We have investigated a special class of RNNs called echo state networks (ESNs) (Jaeger, 2001) for Brain Machine Interfaces (Rao et al. 2005) that have the advantageous properties of being much more practical due to the limited number of adaptive parameters (as compared to unconstrained RNNs). Additionally, they require straight backpropagation (Haykin, 1999) to be trained. They are an alternative technique to TLFNs for testing dynamic modeling of the STLmax parameter or any of the other parameters of interest. Due to the difficulty of the control task, validation of these dynamical models preferably progresses from synthetic models built from coupled dynamical systems simulators (as explained below) to later real STLmax time series taken from a variety of periods (ictal, pre-ictal and post-ictal). Comparisons are made based on the normalized mean square error in a test set using the autonomous mode (i.e., the model is seeded in the trajectory, and the output of the system is feedback to the input). A model that is able to predict longer with an error below a certain value for many different conditions is considered a preferable model.

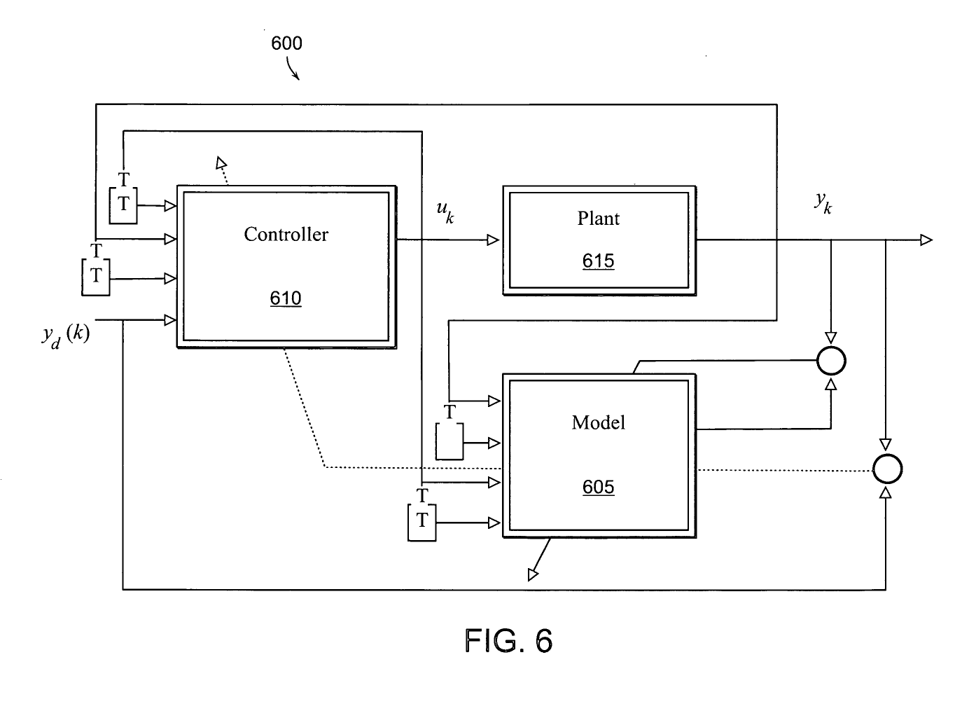

Referring now to an embodiment of a seizure prevention system 600 that incorporates stimulation control based on a model (shown in FIG. 6), after selection of the model 605, the next step is to train the controller 610 in series with the plant 615 to provide a designated STLmax. Because the model 605 is global nonlinear, the controller 610 must also be global and nonlinear, with a similar architecture. Potential problems with this scheme related to the discrete event simulation to the brain, the noise, and the intrinsic delays in the control loop can be addressed with standard procedures of control theory (Narendra 1990).

One of the difficulties of global modeling of nonlinear systems is the possible occurrence of system bifurcations in the controlled path. In this case, the model can become invalid temporally and the expected output is not obtained. This problem may be better handled by local linear modeling, because the latter can handle bifurcations, although a transient will be inevitable.

2. Multiple Switching Local Linear Models (MSLLM).

Some embodiments of the invention incorporate MSLLM models, which address the above problem advantageously by using a “divide and conquer” strategy to simplify the characterization of complex dynamics by dividing the phase space into more or less homogenous regions that can be well modeled by a linear model. One approach incorporates methods we have proposed to control unknown chaotic systems using data-driven multiple models (Cho et al., 2004). The concept of multiple models with switching has been employed in order to simplify both the modeling and the controller design. Multiple models are plausible if a function ƒ to be modeled is complicated because global nonlinear models may not be capable of approximating ƒ equally well across all space. In this case, the dependence on representation can be reduced using local approximation where the domain of ƒ is divided into local regions and a separate model is used for each region. Local linear models are chosen and derived through competition using the Self-Organizing Maps (SOM), as described by Kohonen (1995).

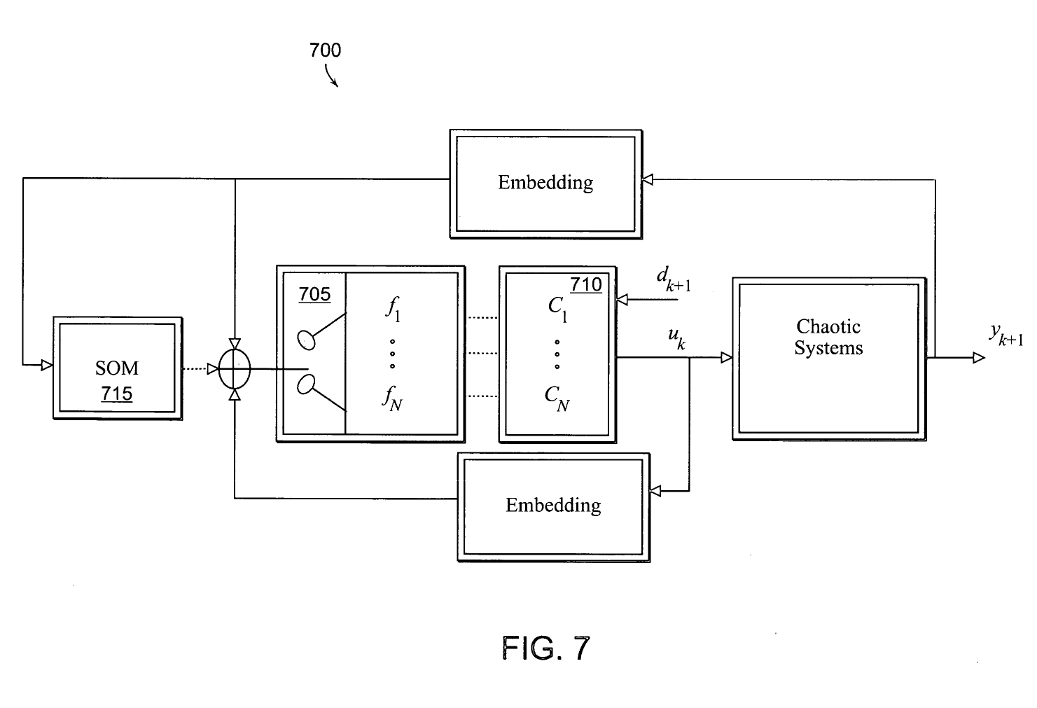

A schematic diagram illustrating a SOM-based multiple controller scheme 700 is shown in FIG. 7. Once the chaotic systems are identified using multiple models, it is necessary to associate these models with corresponding controllers. The associated controller to each of the local linear models can be easily designed a priori. The system identification block 705 has N predictive models, denoted by {ƒᵢ}ᵢ₌₁^{N}, in parallel. Corresponding to each model ƒᵢ, a controller Cᵢ 710 is tuned such that Cᵢ achieves the control objective for ƒᵢ. Therefore, at every instant one of the models is selected and the corresponding controller is used to control the actual system. In this architecture, once the current operating region is determined by the SOM 715, the associated controller is triggered so that the plant tracks the desired signal, dₖ₊₁, as shown in FIG. 7.

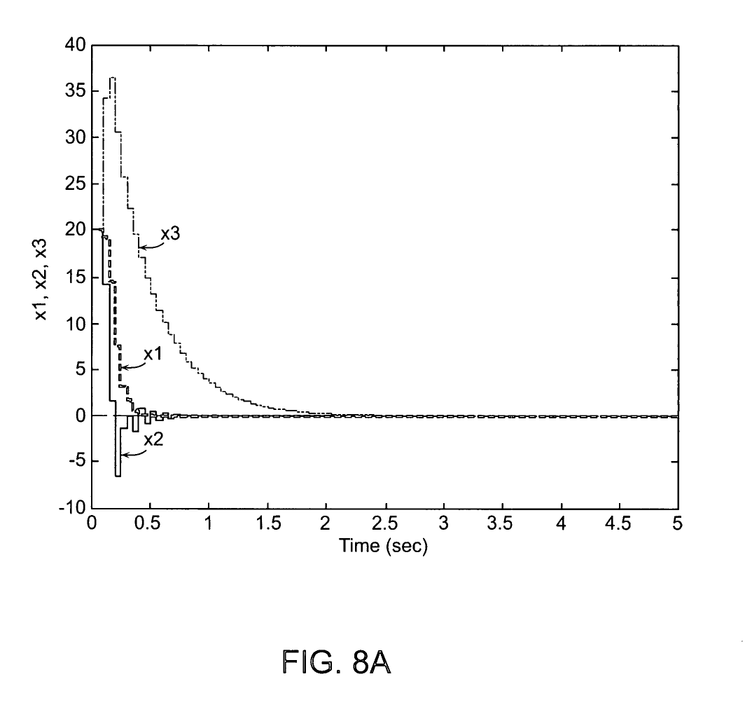

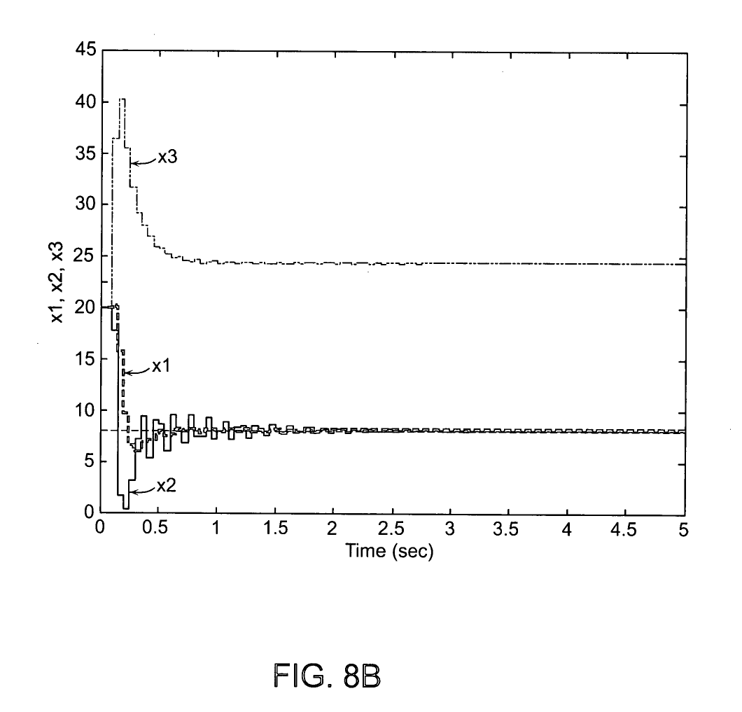

We considered the Lorenz attractor to examine the effectiveness of the proposed controller (i.e., multiple model based sliding mode controller) under the assumption that the plant considered is unknown. Referring to FIGS. 8A and 8B, the results show that the derived control using multiple models is simple and very effective. The plots in FIGS. 8A and 8B show, respectively, stabilizing a saddle steady state (FIG. 8A) and an unstable fixed point (FIG. 8B) of Lorenz system: \dot{x}₁=σ(x₂−x₁), \dot{x}₂=−x₂−x₁x₃+Rx₁+u, \dot{x}₃=x₁x₂−bx₃, with the parameters R=28, σ=10, b=8/3 and 0.05 integral step and 64 linear models.

From the linear model, a controller can be easily derived using the inverse control framework. In several embodiments, the invention applies MSLLM in two basic configurations: one to control directly the STLmax (or other dynamical descriptors such as Kolmogorov entropy, Stationarity index, Pattern Match Statistics, Recurrence Time Statistics, F-Statistics), and the other associated with a data mining strategy applied to the EEG directly.

MSLLM applied to STLmax (or other dynamical descriptors such as Kolmogorov entropy, Stationarity index, Pattern Match Statistics, Recurrence Time Statistics, F-Statistics): This approach utilizes a strategy developed to control unmanned aerial vehicles (UAVs) (Cho et al., 2005). Difficulty in this implementation may be found in the number of linear models and the embedding dimension necessary to properly describe STLmax dynamics or other dynamical descriptors. The controller is designed using the sliding mode approach as described (Cho et al. 2005). We have tested this implementation in nonlinear systems with success. Accordingly, the method is applied directly to the STLmax or other dynamical descriptors, without using further simulators.

Multiple Models with data mining features: After the brain states are classified based on the extracted EEG features (as explained infra), the current state can be identified. From this point we can use sequences of system states of the observation to understand dynamic behaviors, and model the system as a hidden Markov model (HMM). HMMs have been successfully applied to many research areas for the past several years such as speech recognition (Rabiner 1989) and EEG classification (Huang et al. 1996; Obermaier 1999). In EEG classification, the feature's switching time is often used in HMM modeling. Moreover, in one study HMMs were shown to detect non-stationarities, which correspond to the change of states (Penny and Roberts 1998). With the resulting Markov model, the control decision can be obtained from the current state. If the current state is neighbor of a seizure state and the probability of transition from the current state to the seizure is above a threshold, the stimulation output and its parameters are sent to a stimulator. The brain is stimulated with the proper stimulating patterns to move into a safe state.

### Characterizing Dynamical Descriptors of Brain States Associated with Seizures

The invention is based on a strategy of interrupting, delaying, or preventing the occurrence of an impending seizure by altering the dynamical characteristics of brain activity (e.g., EEG) during the preictal state. Successful intervention, provided by the invention in the form of electrical stimulation, necessarily relies on accurate detection of “dynamical descriptors” (quantifiers of EEG signals or the spatiotemporal patterns associated with them) that exhibit detectable changes during the preictal state.

As discussed, and further illustrated in the Examples infra, Short-Term Maximum Lyapunov (STLmax) exponent-based methodology can be effectively used for characterizing the spatio-temporal dynamics in temporal lobe epilepsy. As shown, this methodology is reliable (i.e., sensitive and specific) for detection of the preictal state. In this section we describe various embodiments of the STLmax algorithm suitable for use in closed-loop seizure control systems. Additionally, we describe other dynamical descriptors besides STLmax that can be used and may be more sensitive, specific and practical for this purpose.

1. Variations of Short-Term Maximum Lyapanov (STLmax).

The algorithms demonstrated in the Examples use the same embedding parameters (dimension and delay) throughout the data sets that are tuned to the expected dimensionality of the EEG attractor during seizure. Although in doing so the values of STLmax time series can be directly compared throughout the record including the seizure state, this choice means that the estimation of the STLmax in seizure-free intervals may be biased, since the reconstruction is done in a space smaller than the true one. It is possible to improve this step by estimating the embedding parameters of the attractor (Abarbanel, 1996; Abraham, 1986; Liebert and Schuster, 1989; Iasemidis et al., 1990) in each segment, and find normalization strategies to compensate for the different embedding dimensions.

The computation of the STLmax can be complicated and somewhat time consuming and accordingly it is not very amenable to fast implementations due the search step in the Wolf's algorithm. Other proposals to estimate the largest Lyapunov exponent based on numerical techniques may be more amenable to digital signal processing implementations (see, for example, Rosenstein et al., 1993; Kruel et al., 1993; Kantz, 1994; Eckmann and Ruelle, 1985; Sano and Sawada 1985; Sauer et al., 1998; and Sauer and Yorke, 1999). Recently we have described a method based on a new factorization of the linearized eigenvector matrix that enables the full estimation of Lyapunov exponents and is fast and stable (Pardalos and Yatsenko, 2005). The use of non-overlapping windows to save computation time can result in STLmax profiles that are noisy. The more efficient algorithms as described may permit overlapping of the data windows (33% 45%, 66%) to obtain better resolution in time for the parameter and smoother profiles.

Another characteristic of the STLmax profiles (and even more so of T index analysis) that can be addressed is the quantification of these quantifiers of EEG activity in the space of the electrodes. Indeed, the brain is a dynamical system with spatial extent; therefore the spatial distribution of STLmax time series may contain clinical information relevant to epilepsy, i.e., it may be used to better localize the epileptic focus. One approach is to generate spatial maps of the STLmax, and also of the T index. The tools available from source localization to quantify the changes of dynamical complexity over the brain can be used (Mosher and Leahy, 1999; Xu et al, 2004).

Below are some other dynamical measures that may be applicable in the epileptic state identification process:

2. Kolmogorov Entropy (K).

Kolmogorov entropy and the Lyapunov exponents quantify the chaoticity of an attractor (Kolmogorov, 1954). The Kolmogorov (Sinai or metric) entropy measures the uncertainty about the future state of the system in the phase space given information about its previous states (positions). The Lyapunov exponents (L) measure the average stretching and folding that occurs along different local directions inside an attractor in the phase space. The sum of the positive Lyapunov exponents should give the Kolmogorov entropy. Therefore, both measures quantify the rate (global for K and local for L) of the production of new entropy by the system.

One challenge is the computational complexity in estimating Kolmogorov entropy, and the approximations being presently made. The Kolmogorov entropy (K) is normally estimated through coarse-grained entropy rate techniques (Palus, et al., 1993). These techniques estimate the joint entropy (Hₚ), and the redundancies (Rₚ) (for p=2, the second order redundancy R₂ is the mutual information I) between the components of the vectors in the phase space. The entropy H₁ of one variable Xᵢ¹ is given by:

$${H}_{1} = H ( {X}_{i}^{1} ) = - {\sum }_{{X}_{i}^{1}} p ( {X}_{i}^{1} ) \cdot {log}_{2} p ( {X}_{i}^{1} ) ,$$

where p is the probability mass function of Xᵢ¹. The joint entropy H₂ of the two variables Xᵢ¹ and Xᵢ² (the first two components of a vector-state in the phase space) is given by:

$${H}_{2} = X ( {X}_{i}^{1} , {X}_{i}^{2} ) = - {\sum }_{{X}_{i}^{1}} {\sum }_{{X}_{i}^{2}} p ( {X}_{i}^{1} , {X}_{i}^{2} ) \cdot {log}_{2} p ( {X}_{i}^{1} , {X}_{i}^{2} )$$

where p(Xᵢ¹,Xᵢ²) is the joint probability mass function of Xᵢ¹ and Xᵢ². The conditional entropy of Xᵢ¹ given Xᵢ², that is, the entropy of Xᵢ¹ that remains unaccounted, even after observation of Xᵢ¹ through Xᵢ², is:

$$H ( {X}_{i}^{2} \backslash {X}_{i}^{1} ) = - {\sum }_{{X}_{i}^{1}} {\sum }_{{X}_{i}^{2}} p ( {X}_{i}^{1} , {X}_{i}^{2} ) \cdot {log}_{2} p ( {X}_{i}^{2} \backslash {X}_{i}^{1} )$$

where p(Xᵢ²|Xᵢ¹) is the conditional probability mass function of Xᵢ² given Xᵢ¹. The Kolmogorov entropy is defined as:
Kₚ=H(Xᵢ^{p}\Xᵢ^{p-1}, . . . , Xᵢ¹)=H(Xᵢ^{p},Xᵢ^{p-1}, . . . ,Xᵢ¹)−H(Xᵢ^{p-1}, . . . ,Xᵢ¹)=Hₚ−H₍ₚ₋₁₎

All of this formulation is done for discrete random variables, while in the case of the EEG the amplitude is continuous. Approximating integrals with sums for this case can be problematic, and it may be one of the sources of weak results of the application of Kolmogorov entropy. However, the Computational NeuroEngineering Laboratory has recently proposed a new technique to train adaptive systems called Information Theoretic Learning (Principe et al., 2000) This technique estimates Renyi's quadratic entropy for continuous random variables without ever estimating the probability density function (PDF). This is possible when the Parzen window

$$\hat{f} ( X ) = \frac{1}{N} {\sum }_{i = 1}^{N} k ( X - {X}_{i} )$$

is utilized to estimate the PDF. Indeed, H₂(X)=−logE[fₓ(X)] and using the Parzen estimator we obtain

$${\hat{H}}_{2} ( X ) = - log \frac{1}{{N}^{2}} {\sum }_{j = 1}^{N} ( {\sum }_{i = 1}^{N} {κ}_{σ} ( {X}_{j} - {X}_{i} ) )$$

where K is any kernel that obeys the Mercer conditions (such as the Gaussian kernel). It is therefore quite straightforward to estimate Renyi's entropy of a continuous random variable by substituting H into Ĥ (Erdogmus and Principe, 2002).

The calculation complexities are O(N²), but the recent methods of estimating densities using n-body problems (Elgammal et al., submitted) can perform the calculation in O(N·logN), which makes the method practical for EEG analysis.

3. Stationarity Index.

It is well accepted that the EEG is a nonstationary signal. The nonstationarity of EEG can be defined in terms of its waveforms (i.e., signal patterns), statistical properties (distribution), or the dynamical characteristics in state space (complexity or chaoticity). Advantages of applying the stationarity index to the spatiotemporal properties of EEG include: (1) the assumption of stationarity is not needed, (2) applicability to stochastic or chaotic processes, and (3) faster computation compared to most other dynamical measures. The inventive methods incorporate two stationarity indices and their spatiotemporal patterns related to preictal transitions: Pattern Match Statistics (PMS) and Recurrant Time Statistics (RTS).

3.1 Pattern Match Statistics (PMS).

PMS can be utilized to quantify the stationarity of EEG based on the regularity of the signal patterns. The measure estimates the likelihood of pattern similarity (stationary parts) for a given time series. PMS has been applied for detecting EEG state changes, especially seizures (Shiau, 2001; Shiau et al., 2004). As discussed, major advantages of PMS include the fact that it can be interpreted in both stochastic and chaotic models, and it can be fast computed. The steps to calculate PMS include construction of state vectors, searching for the pattern matched state vectors, and the estimation of pattern match probabilities. Given an EEG signal U={u₁, u₂, . . . , uₙ}, let \hat{σ}ᵤ be the standard deviation of U. For a given integer m (embedding dimension), reconstruct state vectors of U as xᵢ={uᵢ, uᵢ₊₁, . . . , uᵢ₊ₘ₋₁}, 1≦i≦n−m+1, then for a given positive real number r (typically r=0.2\hat{σ}ᵤ), xᵢ and xⱼ are considered pattern matched to each other if:
|uᵢ−uⱼ|<r,|uᵢ₊ₘ₋₁−uⱼ₊ₘ₋₁|<r, and sign(uᵢ₊ₖ−uᵢ₊ₖ₋₁)=sign(uⱼ₊ₖ−uⱼ₊ₖ₋₁) for 1≦k≦m−1

Then

$$PMS = - \frac{1}{n - m} {\sum }_{i = 1}^{n - m} ln ( {\hat{p}}_{i} ) ,$$

where
pᵢ=Prob{sign(uᵢ₊ₘ−uᵢ₊ₘ₋₁)=sign(uⱼ₊ₘ−uⱼ₊ₘ₋₁)|xᵢ and Xⱼ are pattern matched}

As with the calculation of STLmax, PMS is calculated for each sequential 10.24-second non-overlapping EEG segment for each recording channel.

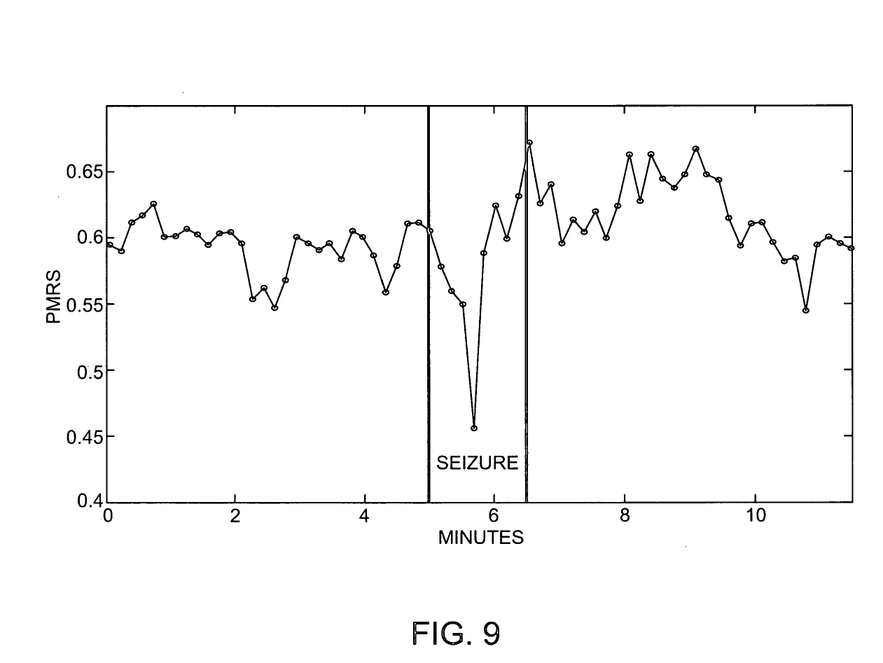

FIG. 9 shows a typical PMS profile before, during, and after a seizure. Referring to FIG. 9, it is seen that the PMS values drop to the lowest point during the seizure, and reach the highest point immediately after the seizure ends. These observations suggest that the EEG signal during the ictal period is less complex than other periods, and that the signal during the postictal period is more complex. Accordingly, methods can be developed using sequential calculations of PMS to detect changes of dynamical states in the EEG signals.

3.2 Recurrence Time Statistics (RTS).

Motivated by simplicity of the calculation, RTS is a measure rooted in nonlinear dynamic theory that can also be used to quantify the stationarity of a signal (Gao, 1999). RTS is defined in the following way. Assuming that we have a scalar time series x(i), i=1, 2, . . . , M, where i is the time index, according to Takens' embedding theorem (Takens, 1981), the corresponding m-dimensional phase space can be constructed from the time series Xₖ=[x(k), x(k+L), x(k+2L), . . . , x(k+(m−1)L)], where L is the time delay. The vector sequence Xₖ, {k=1, 2, . . . , N}, constitutes a trajectory in the phase space with N=M−(m−1)L. Now choose an arbitrary reference point, X₀, in this constructed phase space and consider the recurrence to its neighborhood of radius r:Br(X₀)={X:∥X−X₀∥≦r}. Denote the subset of the trajectory that belongs to Br(X₀) by S₁={Xₜ₁, Xₜ₂, . . . , Xₜᵢ, . . . }. These points are called Poincaré recurrence points, and the Poincaré recurrence time is defined as the elements of {RTS(i)=tᵢ₊₁−tᵢ, iε(1, 2, 3 . . . )}. The value of RST at the reference point X₀ is the mean of this set. Likewise, the whole phase space RTS character is the average of the mean RTS of all the reference points.

To implement RTS on EEG signals, the signal is partitioned into non-overlapping windows of length 10.24 sec. The phase space of each window is constructed accordingly. Furthermore, two parameters need to be decided. They are the embedding dimension m and the time delay L. According to Taken's embedding theory, if the attractor's dimension is D (may be a non-integer) then a constructed phase space with an embedding dimension of m>2D+1 (m should be an integer) is able to reveal the underling dynamics.

For an unknown dynamical system, such as the brain, there is presently no established method to define D. However, many authors concur that the seizure state can be described by a low-dimensional dynamical system (Hively et al, 2000; Lai et al., 2002). The final value of m is determined by examining the simulation results. Initialize D to the smallest number that is larger than the limited cycle correlation dimension (Iasemidis et al, 1999), which is D=1.5. This causes the initial value of m to be 2×1.5+1=4. The value of D is then increased until the performance becomes acceptable. The delay parameter, L, needs to be small enough to capture the shortest change present in the data and large enough to generate the maximum possible independence between components of the phase space vectors. We adopt the method introduced by Abarbanel (1996) to decide this parameter, where the value of L is set to the lag corresponding to the first zero of the autocorrelation of the time-domain EEG. To calculate the autocorrelation, in-seizure EEG samples were used.

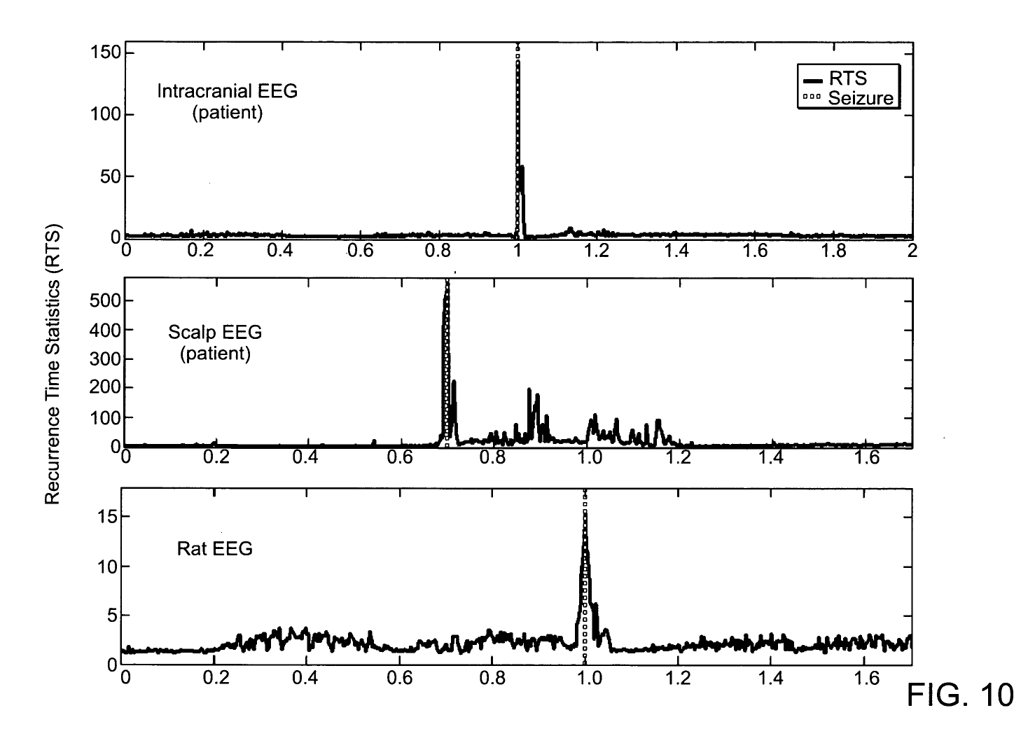

Results from our studies on human and rat EEG signals, (see, e.g., Table 2 and Example 2) show that the RTS exhibits significant change during the ictal period that is clearly distinguished from the background interictal period, as shown in FIG. 10. More particularly, FIG. 10 shows representative RTS profiles before, during, and after a seizure in EEGs recorded intracranially or from the scalps of human patients, or from rats exhibiting spontaneous seizures.

Additionally, through the observations over multi-channel RTS features, the spatial pattern from channel to channel can also be traced. Existence of these spatiotemporal patterns of RTS supports utilization of RTS in automated seizure warning algorithms.

4. Going Beyond the T-Statistics to Characterize the Multidimesional Time Series.

4.1 F-Statistics.

The T-statistic has been extensively utilized in our automated seizure warning algorithms because it is a first step to quantify the spatial dependence of the dynamical measure profiles over time. However, T-statistic can only be applied to quantify the statistical/normalized mean difference of dynamical measures between two electrode sites. In most instances, transitions of the preictal state can be better recognized by studying the spatio-temporal dynamical pattern among a group of electrode sites (n>2). In such cases, directly quantifying statistical effect among an electrode group may be better or more efficient than taking average T-statistics from all electrode pairs among the group. Yang and Carter (1983) checked the efficiency of One-Way Analysis of Variance (ANOVA) with time series data. They considered the problem of testing the null hypothesis of equality of the group means, and demonstrated that, in many practical situations, the usual ANOVA F test, performed on mean of the observations over time, provides an efficient test. Accordingly some embodiments of the algorithms apply ANOVA F-statistics to quantify spatiotemporal dynamical relationships among critical electrode sites for detection of the preictal state.

4.2 Interdependency Measure Between EEG Signals.

Another alternative to T-index is to directly estimate interdependency from a pair of EEG signals. One advantage of such measures over T-index is that they can be more easily interpreted or verified by carefully examining EEG signals. Furthermore, interdependency measures can offer better temporal resolutions since estimation of T-index requires many samples of dynamical measures.

Understanding the interrelations between multiple time-series has numerous applications in signal processing and engineering. Nonlinear dependencies between multiple signals have been studied in the last two decades, but with limited success. Popular methods utilize concepts based on generalized mutual information (Pompe, 1993), and instantaneous phase measures using Hilbert transforms (Hoyer et al., 2000; Rosenblum and Kurths, 1998) and Wavelet transforms (Lachaux, 1999). A difficulty with these methods has been the need to use very long data series and computational complexity due to the handling of this data. Recently, Arnhold et al. (1999) introduced the similarity index technique (SI) to measure asymmetric dependencies between time-sequences that can also identify the information flow direction.

Given two signals, X and Y, the SI is defined as

$$S ( X \vert Y ) = \frac{1}{N} {\sum }_{n = 0}^{N - 1} \frac{{R}^{n} ( X )}{{R}^{n} ( X \vert Y )} .$$

It quantifies the average influence of Y on X. Rⁿ (X) measures the average Euclidean distance between the sample-vector xₙ, which is constructed by embedding the original time series in a delay vector, and its k nearest neighbors in a neighborhood of radius ε, at time instant n. Similarly, the quantity Rⁿ(X|Y) measures the average Euclidean distance between xₙ and the sample-vectors of X whose time indices correspond to the time indices of the nearest neighbors of yₙ. By definition, 0≦Rⁿ(X)≦Rⁿ(X|Y), and the ratio Rⁿ(X)/Rⁿ(X|Y) is always in [0,1]. As a consequence, S(X|Y)=1 implies X is completely dependent on Y. This suggests that recurrence of a state in Y implies a recurrence in X. On the same principles, S(X|Y)=0 implies complete independence between X and Y.

Similarly, it is possible to quantify the average dependence of Y on X by

$$S ( Y \vert X ) = \frac{1}{N} {\sum }_{n = 0}^{N - 1} \frac{{R}^{n} ( Y )}{{R}^{n} ( Y \vert X )} .$$

Comparing S(X|Y) and S(Y|X), we can determine which signal is more dependent on the other. By design, the similarity index can identify nonlinear dependencies. A difficulty with this approach is that at every time instant, we must search for the k nearest neighbors of the current embedded signal vectors among all N sample vectors; this process requires O(N²) operations. In addition, the measure depends heavily on the free parameters, namely, the number of nearest neighbors and the neighborhood size ε. The neighborhood size ε needs to be adjusted every time the dynamic range of the windowed data changes.

### Self-Organizing Map-Based Similarity Index (SOM-SI)

We have previously developed a SOM-SI, as an interdependency measure between signals, as further discussed below. Conceptually, this method relies on the assumption that if there is a dependency between two signals, the neighboring points in time will also be neighboring points in state space. Since this requires searching for the nearest neighbors in the state space (formed by embedding) for large data sets, the computational complexity can become unmanageable. However, a self-organizing map (SOM) based improvement to this method can reduce computational complexity, while maintaining accuracy as follows. By mapping the embedded data from signals onto a quantized output space through a SOM specialized on these signals, and utilizing the activation of SOM neurons to infer about the influence directions between the signals, this can be achieved in a manner similar to the original SI technique.

The SOM-SI algorithm is designed to reduce the computational complexity of the SI technique. The central idea is to create a statistically quantized representation of the dynamical system using a SOM (Haykin, 1999; Principe et al., 2000). For best generalization, the map needs to be trained to represent all possible states of the system (or at least with as much variation as possible). As an example, if we were to measure the dependencies between EEG signals recorded from different regions of the brain, it is necessary to create a SOM that represents the dynamics of signals collected from all channels. The SOM can then be used as a prototype to represent any signal recorded from any spatial location on the brain, assuming that the neurons of the SOM have been specialized in the dynamics from different regions.

One of the salient features of the SOM is topology preservation (Haykin, 1999; Principe et al., 2000); i.e., the neighboring neurons in the feature space correspond to neighboring states in the input data. In the application of SOM modeling to the similarity index concept, the topology preserving quality of the SOM enables us to identify neighboring states of the signals by neighboring neurons in the SOM. Details for calculating SOM-based SI can be found, for example, in Hegde et al., 2003.

The computational savings of the SOM approach is an immediate consequence of the quantization of the input (signal) vector space. The search for nearest neighbors involves O(Nm) operations, as opposed to the O(N²) of the original algorithm, where N is the number of samples and m is the number of neurons in the SOM (m<<N by design).

From studies on simulation and on EEG signals, we observed that the SOM-based SI is not significantly different from the values obtained from the original algorithm. Secondly, we found that synchronization quantified by SI increases momentarily a few minutes prior to a seizure and stays high during the seizure, and that there is a sudden drop followed by a sharp increase after the seizure. This pattern in SI values was observed in all seizures analyzed. Therefore, this spatiotemporal pattern in SOM-based SI is incorporated into some embodiments of the algorithms of the automated seizure warning systems.

### Data-Mining Methods for Characterizing EEG States

In this section we briefly describe approaches from data-mining and classification to characterize EEG states that can be then used in a multiple model control framework described above.

Brain activity cannot be described mathematically with the present state of knowledge. Furthermore, only a subset of brain states is measurable with a finite number of sensors utilized. The inventive methods are based on an assumption that observations generated from the same or similar set of system parameters have analogous behaviors. With limited knowledge and information, this modeling task can be accomplished by extracting the crucial features from the EEG. It is known that many kinds of features can be extracted from the brain such as parametric modeling (Pardey et al, 1996) and complexity measures (Rezek and Roberts, 1998). Moreover, the brain can be modeled indirectly by clustering groups of EEGs that have similar properties or are located in the same area in feature space. Thus, EEGs generated from the same brain state will belong to the same neighborhood in feature space.

Here we introduce several data-mining techniques useful to analyze the EEG data. The main concept is to observe the EEG data using sliding windows of suitable size. These short segments of the whole EEG time series are analyzed using the following steps: Feature Extraction, Clustering, and Classification.

1. Feature Extraction.

Dynamics and other indicators are used to extract suitable information and represent each window. Several features can be extracted from a time series. Dynamical systems analysis tools and others statistical measurements can be applied to analyze EEG data. More specifically, Lyapunov exponents, Complexity, Spikes Representation, and statistical reduction methods such as Independent Components Analysis can be used.

1.1 Lyapunov Exponents.

See description, supra.

1.2 Complexity.

For short and noisy time series correlation dimension, as other measures, can fail. In an EEG signal, what must be analyzed is how the system is changing among different situations as a pre-seizure system or other situations. In those cases the problem is to obtain, for short time lags, an estimation of the desired measure, because the system is always changing and just short segments can be considered as belonging to the same system. To analyze the changing of the complexity of a system, it can be useful to obtain an on-line estimation of the correlation dimension (Christian and Lehnertz, 1998) to analyze the transition of the system.

1.3 Spikes Representation.

Using spikes representation, the time series can be represented as a pattern of three digits (+1, −1, 0). One simple method to obtain this representation is thresholding the EEG signal according to some suitable amplitude values (Lewicki, 1998).

1.4 Statistical Dimension Reduction.

Classical statistical techniques as PCA or ICA (Pierre, 1994) can be applied to obtain a dimension reduction. These techniques capture in a lower dimension the part of the signal with more information. While in PCA the goal is to obtain uncorrelated variables that minimize the loss of information, in ICA statistical independent components are calculated.

2. Clustering.

Once a time series has been represented by the features extracted in the previous step, clustering methods are applied to observe how different parts of the EEG time series are grouped together. Once all the indicators of the signal have been calculated, the short time windows of the EEG data can be represented as pattern vectors where each element corresponds to one of the indicators. Partitioning and hierarchical clustering methods are applied on these elements to obtain suitable clusters. Applying these methods makes it possible to analyze which parts of the whole EEG signal can be considered similar. Obtaining good quality clusters is an important step. In fact, if groups show strong and dominant similarity it is possible to consider them as different states of the brain. Investigating how many states of the brain exist and the relations between them is a crucial step. In fact, this information will provide the basis of the Markov model to of the probability of change states.

Clustering has been characterized as unsupervised learning in which data is analyzed without a priori knowledge (Han and Kamber, 2001). The data objects are clustered or grouped based on the principle of maximizing the interclass similarity. Clustering can also facilitate taxonomy formation, that is, the organization of observation into a hierarchy of classes that group similar events together. Many clustering algorithms have been proposed and the effectiveness of the methods can depend on the characteristics of data domains. In general, major clustering methods can be classified into partitioning and hierarchical methods. A partitioning method divides the data objects into k groups, which satisfy an optimal criterion that the objects in the same class are closely related to each other and objects of different classes are dissimilar. A hierarchical method creates a hierarchical decomposition of the given set of data objects.

In the agglomerative approach to hierarchical clustering, each cluster initially contains exactly one sample. Next, two clusters which are most similar are merged into one cluster, and this step is repeated until one big cluster is formed. The most natural illustration of hierarchical clustering is a dendrogram representing a nested grouping pattern (Han and Kamber, 2001; Duda et al., 2001). Depending on where the cut t is done, a different clustering of the data is obtained.

The similarity measures obtained when two clusters are merged together can be used to determine whether groupings are natural or forced. Hierarchical clustering can give various levels of clustering structures; additionally, it can help to identify subgroups or patterns of interest by using nested grouping structures. For example, if a set of data constructed from a preictal period is grouped together and some subgroups in the cluster show strong dominant similarity, then it can be useful to identify those patterns which can be significant indicators of seizure occurrence, or different states of the brain.

3. Classification.

Once the clustering structure has been determined and labels can be applied on the data, classification methods are used to obtain a model that assigns each unknown element to a particular class. Classification, or pattern recognition, is the process of finding a set of models which describes or distinguishes data groups. The formation of models is supervised because is based on a set of data objects whose class labels are known. In this case the class label is determined in the clustering phase. Once a clustering structure is adequately detected and the clusters indicate different behaviors or states of the brain, the discriminant power of the formed structure is measured by classification, assigning to the data the cluster label at which belongs. By combining the hierarchical clustering and classification, the intrinsic grouping structure with the most discriminant power can be found. Further, a classification problem is solved to recognize at which state a new data point belongs. This information is used as feedback to select the correct stimuli to apply.

For this purpose, we apply well-known pattern recognition algorithms called Support Vector Machines (SVMs) (Vapnik, 1995; Burges, 1998). SVMs are recent methods widely used for classification problems and are considered the state-of-the-art methods for binary classification.

For the development of new models, after the modeling phase is completed, the model is verified. The mapping of EEG into feature space is analyzed to ensure that incorrect mapping of the EEG state cannot cause an error in controlling the seizure. If a model is not correct, the modeling phase is repeated. In this case, the features of the EEG are reconsidered to obtain the stable feature space that corresponds to the EEG activity.

### Design of Control Models for State-Dependent Seizure Prevention Devices

In the state-dependent automated seizure prevention systems of the invention, dynamical descriptors of brain state (STLmax and others, as described above) are estimated in real-time from several brain sites, and used as the observable output for the closed-loop control scheme, as shown schematically in FIG. 1. The stimulating input to the brain is composed of electric biphasic pulses applied to different brain structures. As discussed, the design of the state-dependent control model is based on the assumption, supported by studies described herein, that when a preictal state is detected, electrical stimulation of brain regions changes the brain's spatio-temporal dynamics and can effectively control temporal lobe epileptic seizures.

The main task in designing a control model is to find optimal stimulation parameters that give the best control performance. Performance of the system can be defined in two ways. “Acute effect” refers to the ability to change the suspected preictal state back to the normal interictal state with respect to specific dynamical patterns. “Chronic effect” refers to the ability to change the seizure patterns (e.g., seizure frequency, seizure severity, etc). Since these two performances are believed to be related under the above assumption, the design of the control model is based on the outcome of acute effects. One way to quantify the acute effect, i.e., one performance measure, is to measure the duration between the time of stimulation and the time when the dynamical pattern moves back to a normal state. However, other performance measures such as the time interval to the next seizure can also be considered.

After selection of a control model, an ASW system can be tested using control and experimental periods, for example in an animal model of epilepsy. More specifically, after training the ASW system in a control period, optimal parameter settings for detecting the preictal state are determined. In addition, mean performance measure (and standard deviation) is observed during the control phase. These control performance outcomes are then used for comparisons with those obtained in a subsequent intervention experimental phase (IEP).

During the IEP, when an ASW system detects a preictal state, electrical stimulus is given with different combinations of parameters (stimulation treatments). Candidates for parameter combinations for each ASW system can be determined based on the safety consideration and the experience gained from dynamical response studies. Examples of the ranges of stimulation parameters are shown in Table 1.

TABLE 1
Ranges of Stimulation Parameters.

| | |
|---|---|
| Current Intensity | 50 μA - below AD threshold |
| Frequency | 50-400 Hz |
| Duration | 1-30 seconds |
| Pulsewidth | 50-50 μseconds |

Suitable control periods and IEPs using a rat model of epilepsy, and outcomes showing detection and intervention of seizures using a state-dependent ASW in accordance with the invention are described in further detail in Examples below.

### EXAMPLES

The invention is further illustrated by reference to the following non-limiting examples.

### Example 1—Materials and Methods

The following materials and methods can be used as needed generally to practice the invention and to conduct studies as outlined in the Examples below.

1. Rodent Model of Self-Sustained Limbic Status Epilepticus.

Young adult male 200-250 g Sprague-Dawley rats (age approximately 40 days) are anesthetized with isoflurane in oxygen and placed in a Kopf stereotacetic frame. The scalp is split and all soft tissue loosened from the dorsum of the skull. Bipolar insulated stainless steel electrodes are placed bilaterally in the posterior ventral hippocampus for stimulation and recording (from bregma AP −5.3, ML 4.9, DV −5.0 from dura, bite bar −3.3), as described by Paxinos and Watson (1998). The presence of a second electrode also enhances the likelihood of detecting a seizure. Additional monopolar reference and ground electrodes are placed over the cerebellum. All electrodes, intracerebral and reference, are attached to Amphenol connectors and secured to the skull with jeweler's screws and dental acrylic. Animals are allowed to recover for 1 week before experiments are started.

Induction of status epilepticus (chronic hippocampal stimulation): One week following surgery, the after-discharge threshold (ADT) is determined, using 10 second trains of 50 Hz, 1 ms bipolar square waves with an initial current intensity of 60 mA. The intensity is increased by 10 mA steps to 110 mA and by 20 mA steps until a maximum of 250 mA is reached. Preferably, animals with ADTs greater than 250 mA are not studied further to ensure uniformity among the animals with regard to ADT and stimulus intensity.

The protocol for inducing self-sustained limbic status epilepticus has been developed to give a high yield in adult unkindled animals. Animals are stimulated “continuously” for 90 minutes using 10 sec trains of 50 Hz 1 ms bipolar square waves, delivered every 12 seconds. Current is set using an empiric formula established to deliver suprathreshold stimuli. For rats with ADTs of 160 mA or less, the stimulus intensity is about 400 mA; for ADTs of 200 or less, about 500 mA; and for ADTs of 250 or less, about 600 mA. The use of these suprathreshold stimulus intensities ensures that post-ictal refractoriness is overridden and that status epilepticus develops. After 90 minutes, continuous stimulation is stopped and hippocampal activity is recorded for a minimum of 8 hours to ensure that a prolonged period of continuous EEG seizure activity is maintained, and to determine the efficacy of stimulation.

The evolution of the EEG is evaluated according to a standard 5-point scale, and the animals are categorized by the most “advanced” stage reached as follows: 1—interictal; 2—intermittent discrete seizures; 3—continuous “high frequency”, greater than 1 Hz; 4—periodic epileptiform discharges with superimposed high frequency ictal discharges; 5—periodic epileptiform discharges only—(PEDS). Previous work has established that choosing animals that have continuous seizure activity (EEG score 3 or higher) for at least six hours after stimulation ensures a uniform risk for the eventual development of limbic epilepsy. Animals that do not meet the EEG criteria preferably are not used, as their chances of developing chronic epilepsy are extremely low.

2. Recording Protocol.

This section describes the general methods for prolonged EEG recordings (Feng, 2000). Although there are some additions or variations in specific experiments according to their purpose, these techniques form a suitable basis for prolonged monitoring. Following the induction of limbic status epilepticus, rats are first placed in standard laboratory housing; however, during the monitoring phase, they are preferably housed in specially designed cages that allow full mobility of the animals, good visualization for video monitoring and a stable recording environment. Each rat is separately housed in a 10-inch diameter cast acrylic tube that is 12 inches high and has a plastic mesh floor. Access to food and water is freely available. Cage illumination is according to a standard 12-hour light dark cycle.

The animals are connected via a 20 cm cable (5 wire shielded) to a swivel electrical commutator which is hard-wired to an EEG recording station. The use of the commutators and the shielded cables as described is critical to the reduction of activity-induced artifact and the preservation of the headsets.

Continuous electroencephalographic activity is recorded daily for 24 hours per day using a commercial video/EEG instrument (Tucker-Davis Technologies, Alachua, Fla.; Monitor, Stellate Systems, Montreal). Continuous electroencephalographic activity is recorded daily using a time-locked video digital electroencephalogram instrument. All recordings are analyzed for the distribution of spike and sharp wave discharges, and their relationship to sleep state, seizure activity, and to the ictal onset region. All recordings are carried out about 14 days after surgery on rats moving freely in a test cage containing food and water.

Video and digital electroencephalographic tracings are time-locked for analysis. The saved EEG records are transferred over a local network to a central computer that serves as an EEG reader. All data are reviewed at an offline reading station consisting of a computer that is connected to the vivarium computers via a local network. The EEG segments are reviewed on the computer monitor. When a saved EEG sample is found to be a true seizure, the time of occurrence and duration of the seizure is noted.

Data is analyzed using OpenEXx software (Tucker-Davis). In addition, seizures can be recorded and documented using a commercial computerized seizure recognition program (Monitor, Stellate Systems, Montreal). The online computer seizure recognition system is used to provide critical data reduction by selecting only those segments of the pre hour recordings that are likely to contain seizures.

Data described in Examples below indicate that it is technically feasible to implant electrodes in small animal brains such as those of rats. Data acquisition studies also described below have confirmed that reproducible digital electroencephalographic information can be obtained using the experimental setup as described.

3. Seizure Determination.

Criteria for identifying a behavioral seizure can be as follows: A behavioral seizure score (BSS) is determined using the standard Racine scale (i.e., 0, no change; 1, wet dog shakes; 2, head bobbing; 3, forelimb clonus; 4, forelimb clonus and animal rearing; 5, rearing and falling). The BSS, which is an indirect measure of the amount of brain involved in seizure activity, is equated with seizure spread.

Electrographic seizures in limbic epilepsy rats are usually characterized by the paroxysmal onset of high frequency (greater than 5 Hz) increased amplitude discharges that show an evolutionary pattern of a gradual slowing of the discharge frequency and subsequent post-ictal suppression. In some instances, the seizure begins with high amplitude spikes or polyspikes, followed by a brief period of electrographic suppression. The evolutionary pattern and post-ictal suppression are key elements in determining that an event was a seizure, as artifact (especially head scratching) can have a similar appearance but lacks all of these characteristics.

The electroencephalographic criteria for identifying an EEG seizure are as follows: 1)

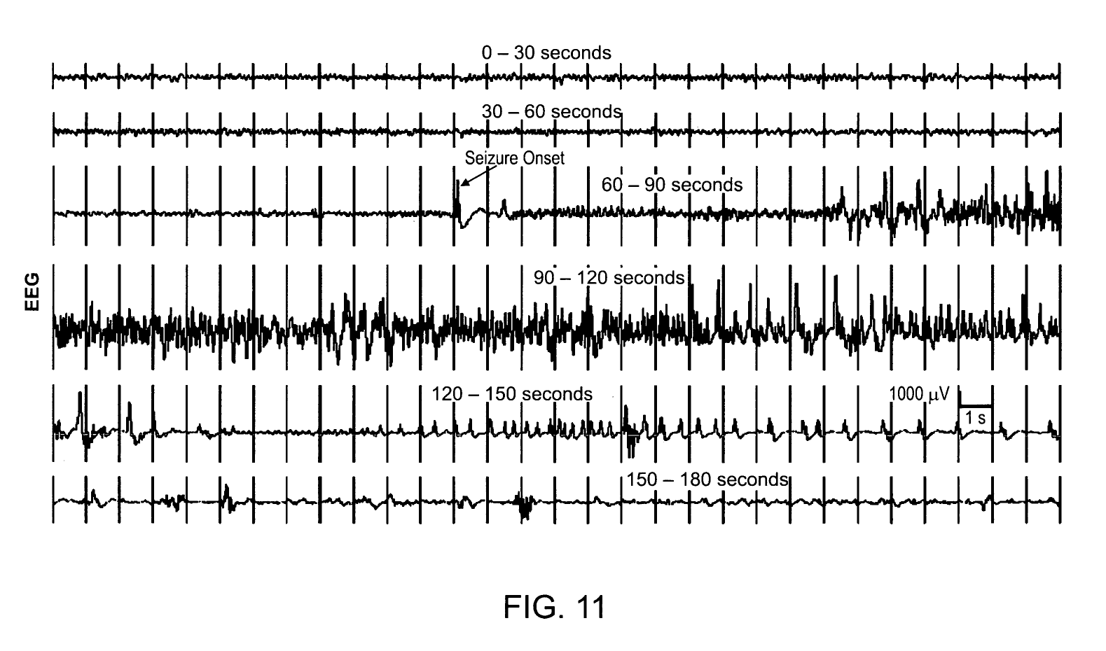

The occurrence of repetitive spikes or spike-and-wave discharges recurring at frequencies>1 Hz, or continuous polyspiking; 2) spike amplitude greater than background activity; and 3) duration of continuous seizure activity greater than 10 sec. FIG. 11 is a graph showing EEG tracings of a representative limbic seizure event obtained in a 65 day old male Sprague Dawley rat following induced status epilepticus. More particularly, FIG. 11 illustrates three minutes of EEG data (demonstrated by 6 sequential 30-second segments) recorded from the left hippocampus, showing a sample seizure from an epileptic rat. This seizure was accompanied by a grade 5 behavioral seizure (Carney, 2004). Distinct rhythmic spike, spike/wave, and polyspike complexes are observed from the right hippocampal electrode recording site (Carney et al., 2004; Nair et al. 2005).

4. Identification of EEG Spatiotemporal Dynamical Features Associated with Preictal State.

Detectable changes in EEGs that can be observed during the preictal state are used to identify spatiotemporal dynamical features associated with the preictal state, which can be used to control the automated seizure warning (ASW) systems. Preictal changes in each dynamical measure may be observed from individual EEG channel (temporal patterns) or from interactions among EEG channels (spatiotemporal patterns), and typically are identified by retrospective analysis of EEGs, recorded approximately in a 2-3 hour interval before a seizure.

After being identified as a candidate for use in an ASW system, the sensitivity and specificity of each preictal pattern is statistically evaluated on long-term continuous EEG recordings from experimental animal studies, for example using CLE rats as described above. There are two phases in the process of statistical evaluation: training phase and experimental phase.

1. Training phase (TP): The purposes of this phase are to determine, for each ASW system, the optimal seizure warning parameters involved in the algorithm and to identify the most critical groups of recording sites. The average duration of the training phase is approximately 2 weeks, with at least 5 seizures recorded (typically CLE rats have an average of 2-3 seizures/week). The determination of optimal parameters is based on the detection ROC curve (described infra) with respect to the sensitivity and false warning rate (per hour) of the algorithm. After the rats have experienced five seizures, the experimental phase commences.

2. Experimental phase (EP): The EP for each rat can last, for example, for about four weeks (during which time rats are expected to have an average of 10 seizures). For each test AWS, the parameter settings are fixed based on the results in the TP. Details of the evaluation procedure is described in Examples below. Typically, a satisfactory ASW system will have the following characteristics: with a 2 hour prediction horizon—at least 80% seizure warning sensitivity, with false warning rate (specificity) of no more than 1 per 8 hours, and overall seizure warning ROC area (AAC) significantly smaller than naïve seizure warning schemes (periodic and random). In some applications of the closed-loop seizure control systems, however, high seizure warning sensitivity may be considered more important than low false warning rate, depending on the stimulation parameters and the responses from the subject. In such cases, high sensitivity (e.g., >95%) with superior performance to naïve seizure warning schemes may be considered a preferable cutoff.

### Example 2—Methods Using EEGs from Human Subjects and Rat Model of Epilepsy for Evaluation of ATSWA for Seizure Prediction

This Example describes the characteristics of epileptic human and animal subjects and EEGs derived from these subjects used for testing an ATSWA system in accordance with the invention.

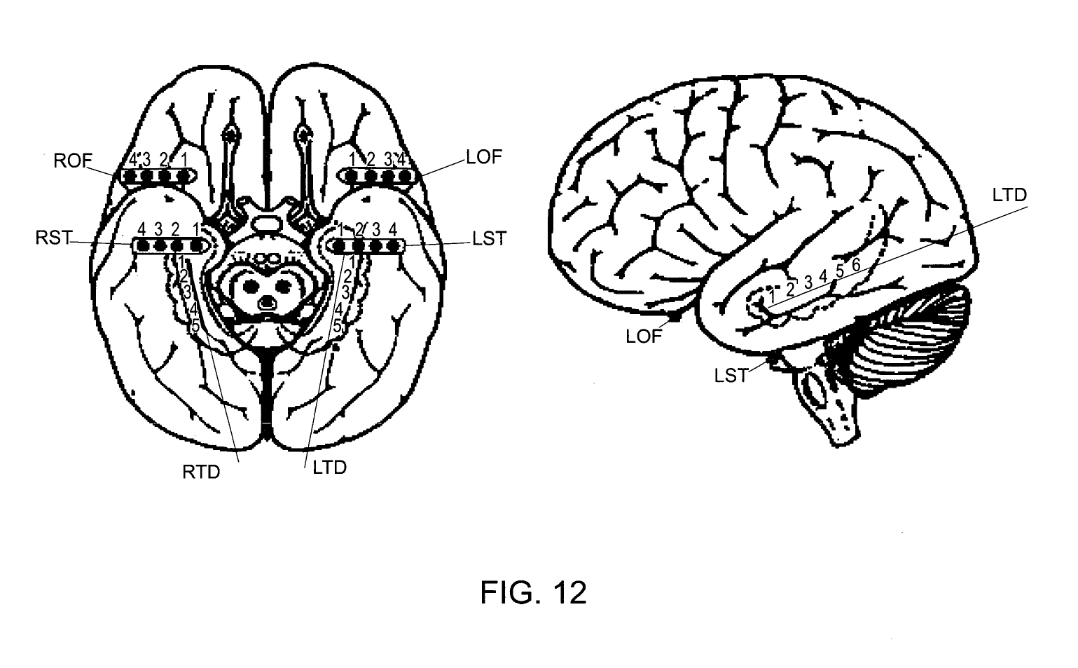

In one series of studies, an adaptive threshold seizure warning algorithm (ATSWA) of the invention was tested in a sample of 18 pre-recorded long-term continuous intracranial (N=10) and scalp (N=8) EEGs. These recordings had been previously obtained for clinical diagnostic purposes. Long-term (3.18 to 13.45 days) continuous recordings were made in these subjects using either multi-electrode EEG signals (28 to 32 common reference channels for intracranial recordings) or signals from 22 channels for scalp recordings (International 10-20 System of electrode placement). The placement of the intracranial recording electrodes is shown in FIG. 12. The positions of the subdural electrodes are shown in the diagram on the left of FIG. 12, and the placement of the depth electrodes is shown on the right. As indicated, subdural electrode strips are placed over the left orbitofrontal (LOF), right orbitofrontal (ROF), left subtemporal (LST), and right subtemporal cortex (RST). Depth electrodes are placed in the left temporal depth (LTD) and right temporal depth (RTD) in order to record hippocampal EEG activity.

In the studies summarized in Table 2, infra, between 6 and 18 seizures of mesial temporal onset were recorded for each patient during the period of recordings. A total of 206 seizures over 144.18 days was recorded with a mean inter-seizure interval of approximately 14.81 hours. All EEG recordings were viewed by two independent board-certified electroencephalographers, to determine the number and type of recorded seizures, seizure onset and end times, and seizure onset zones.

Table 2 also includes data from testing of ATSWA in EEG recordings made in CLE rats, which exhibit spontaneous seizures, as described above. The system was tested in long-term continuous 4-channel intracranial EEG recordings obtained from 5 CLE rats. Recordings from these rats were selected based on duration of recordings (at least two weeks), and number of seizures (at least 5 seizures). Between 7 and 15 grade 5 seizures were recorded for each rat during the period of recordings. A total of 48 seizures over approximately 95 days was recorded with a mean inter-seizure interval of approximately 50 hours.

The characteristics of the datasets from the human subjects and the CLE rats are shown in Table 2.

TABLE 2
Summary of Analyzed EEG Data from Human Subjects and Epileptic
Rats.

| | | | | Mean |
|---|---|---|---|---|
| | | Duration | Range of | Inter- |
| | Number of | of EEG | Inter-seizure | seizure |
| Type of | Patients/Rats and | recordings | interval | interval |
| Recordings | seizures | (days) | (hours) | (hours) |
| Intracranial | 10 patients | 87.5 | 1.52˜119.70 | 13.39 |
| EEG from | with a total of 130 | | | |
| Patients | seizures | | | |
| Scalp EEG | 8 patients | 56.7 | 2.03˜93.91 | 12.82 |
| from | with a total of 76 | | | |
| Patients | seizures | | | |
| Intracranial | 5 rats with a | 95.0 | 2.82˜217.70 | 49.70 |
| EEG | total of 48 seizures | | | |
| from Rats | | | | |

### Example 3—Evaluation of Performance of ATSWA

To evaluate the prediction accuracy of any prediction scheme, a parameter termed a “prediction horizon (PH),” also referred to as the “alert interval” (Vere-Jones, 1995), is used. This is necessary due to the impracticality of predicting the exact time when an event will occur. The PH has been defined as “the time left from the processing window to the unequivocal EEG onset of the seizure” (Litt and Echauz, 2002). After the issue of a warning, a prediction is considered as correct if the event occurs within the preset PH. If no event occurs within the window of the PH, the prediction is classified as a false prediction. The merit of a prediction scheme for a given prediction parameter is then evaluated by its probability of correctly predicting the next event (sensitivity) and its false prediction rate (FPR) (specificity). An ideal prediction scheme should have a sensitivity of 1, and a specificity of zero.

The unit of FPR used in this Example is per hour, and FPR is estimated as the total number of false predictions divided by total number of hours of EEG analyzed. In this Example, ATSWA was evaluated by considering PH of 0.5, 1, 1.5, 2, 2.5 and 3 hours for patient datasets, and 1-6 hours for rat datasets. Analysis in different PH not only can help in assessing the performance/utility of the algorithm for different clinical application but also can enhance the understanding of the optimal PH that is most superior to “naïve” prediction schemes. Tables 3-5 infra summarize the seizure warning performance of ATSWA when sensitivity is at least 80% for each test subject with PH=2.5 hours (=3 hours for rats).

Patients with intracranial EEG recordings. With 2.5 hours PH, an FPR of 0.124 per hour (approximately 1 false prediction per 8 hours) was observed for ATSWA when a sensitivity of 80% or better was required for each patient. The mean prediction time (i.e., the average of the period from the true warnings issued by the algorithm up to the onset of the subsequent seizures) for each patient ranged from 24.6 to 103.6 minutes, with an overall mean 63.8 minutes (Table 3).

TABLE 3
Prediction Performance of ATSWA Algorithm on Patients with
Intracranial Recordings.

| | | | Mean |
|---|---|---|---|
| | | False | Prediction Time |
| Patient | Sensitivity | Prediction Rate | (mins) |
| I-1 | 11/13 = 84.6% | 0.086/hr | 24.6 (±34.1) |
| I-2 | 5/6 = 83.3% | 0.086/hr | 103.6 (±27.7) |
| I-3 | 5/6 = 83.3% | 0.123/hr | 30.1 (±7.7) |
| I-4 | 14/17 = 82.4% | 0.073/hr | 57.2 (±42.1) |
| I-5 | 12/14 = 85.7% | 0.150/hr | 61.5 (±44.4) |
| I-6 | 12/15 = 80.0% | 0.115/hr | 71.6 (±49.9) |
| I-7 | 6/7 = 85.7% | 0.082/hr | 74.0 (±49.5) |
| I-8 | 14/16 = 87.5% | 0.131/hr | 84.1 (±45.4) |
| I-9 | 14/16 = 87.5% | 0.172/hr | 70.6 (±53.1) |
| I-10 | 8/10 = 80.0% | 0.112/hr | 62.4 (±32.9) |
| Total | 101/120 = 84.2% | 0.124/hr | 63.8 (±43.3) |

Patients with scalp EEG recordings. With 2.5 hours PH, an overall FPR of 0.128 per hour (approximately 1 false prediction per 7.8 hours) was achieved in this group of patients when a sensitivity of 80% or better was required for each patient. The mean prediction time for each patient ranged from 27.7 to 97.8 minutes, with an overall mean 65.7 minutes (Table 4).

TABLE 4
Prediction Performance of ATSWA Algorithm on Patients with Scalp
Recordings.

| | | | Mean |
|---|---|---|---|
| | | False | Prediction Time |
| Patients | Sensitivity | Prediction Rate | (mins) |
| S-1 | 8/9 = 88.9% | 0.123/hr | 77.0 (±51.3) |
| S-2 | 8/9 = 88.9% | 0.084/hr | 52.8 (±26.3) |
| S-3 | 7/8 = 87.5% | 0.113/hr | 73.6 (±45.8) |
| S-4 | 8/10 = 80.0% | 0.110/hr | 80.6 (±29.7) |
| S-5 | 10/12 = 83.3% | 0.175/hr | 61.3 (±44.0) |
| S-6 | 4/5 = 80.0% | 0.101/hr | 54.6 (±28.5) |
| S-7 | 7/8 = 87.5% | 0.067/hr | 27.7 (±24.5) |
| S-8 | 6/7 = 85.7% | 0.141/hr | 97.8 (±29.0) |
| Total | 58/68 = 85.3% | 0.128/hr | 65.7 (±37.3) |

Epileptic rats with intracranial EEG recordings. With 3 hours PH, an overall FPR of 0.116 per hour (approximately 1 false prediction per 8.62 hours) was observed.

The prediction time for each animal subject ranged from 33.0 to 91.3 minutes with an overall mean of 69.5 minutes (Table 5).

TABLE 5
Prediction Performance of ATSWA Algorithm on Rats with Intracranial
Recordings.

| | | | Mean |
|---|---|---|---|
| | | False | Prediction Time |
| Subject | Sensitivity | Prediction Rate | (mins) |
| R-1 | 5/6 = 83.3% | 0.083/hr | 55.9 (±39.4) |
| R-2 | 6/7 = 85.7% | 0.100/hr | 91.3 (±61.0) |
| R-3 | 8/9 = 88.9% | 0.114/hr | 33.0 (±33.5) |
| R-4 | 6/7 = 85.7% | 0.158/hr | 76.5 (±58.7) |
| R-5 | 12/14 = 85.7% | 0.141/hr | 85.2 (±43.8) |
| Total | 37/43 = 86.1% | 0.116/hr | 69.5 (±47.1) |

### Example 4—Development and Testing of On-Line Real-Time ATSWA Software

The ATSWA algorithms are written in C++ programs. They include an interface such that it can be placed on the network of the Epilepsy Monitoring Unit (EMU) and continuously read in EEG signals from the Nicolet BMSI™ 6000 recording systems used in the EMU. The system pilot set up and testing of the hardware configuration was accomplished using a dedicated computer as the “analysis computer”, and the Nicolet BMSI™ 6000 as the EEG recording system.

The software was first tested by simulating multiple recording sessions over a 3-day period, using a sine wave generator as the signal input. Subsequently, the system was successfully tested in four pilot studies involving patients admitted to our medical facility's Epilepsy Monitoring Unit for clinical diagnostic procedures. A similar configuration was also set up in our animal laboratory by interfacing with the Stellate EEG recording system. This system is used on-line in real-time to test and refine the ATSWA algorithms.

Statistical evaluation of seizure warning performance of ATSWA. Without a standard EEG database, it is difficult to conduct objective comparisons between the performance of ATSWA and those reported from other automated seizure warning algorithms. A current consideration is how EEG-based seizure warning systems may be statistically validated (Andrzejak et al., 2003). As one stage of evaluation, we compared the performance of ATSWA with those obtained from two statistical derived naïve seizure warning schemes that do not utilize information from the EEG signals—periodic and random seizure warning algorithms. The periodic and random prediction schemes are simple and intuitive. The periodic scheme predicts with a fixed time interval. The random prediction scheme predicts events according to an exponential distribution with a fixed mean.

Periodic Warning Scheme: In the periodic prediction scheme, the algorithm issues a seizure warning at a given time interval T after the first seizure. For each subsequent warning, the process is repeated. As with the other algorithms, the warnings within the same PH from the preceding warning were ignored. Runs with a broad spectrum of T values were performed on all data from all patients and rat subjects described above.

Random Warning Scheme: This algorithm first issues a warning at an exponential distributed (exp(l)) random time interval with mean l, after the first seizure. After the first warning, another random time interval is chosen from the same distribution to issue the next warning. This procedure is repeated after each warning. Similarly, the warnings within the same PH from the preceding warning were ignored. Runs with a broad spectrum of l values were performed on all data from all patients and rat subjects.

Generating seizure warning ROC curves: One can compare any two prediction schemes by their sensitivities at a given FPR, or conversely, compare their FPRs at a given sensitivity. However, in practice it is not always possible to fix the sensitivity or FPR in a sample with a small number of events. Moreover, there is no universal agreement on what is an acceptable FPR or sensitivity. One can always increase the sensitivity at the expense of a higher FPR. A similar situation occurs in comparing methods of disease diagnosis where the tradeoff is between sensitivity, defined as probability of a disease being correctly diagnosed, and specificity, defined as the probability of a healthy subject being correctly diagnosed.

A common practice in comparing diagnostic methods is to let the sensitivity and the specificity vary together (by changing a parameter in a given prediction scheme) and use their relation, called the receiver operating characteristic (ROC) curve, to evaluate their performance.

In this Example, we estimate the warning ROC curve for each test algorithm in each patient. The prediction parameters used for the construction of each ROC curve were: the distance D between UT and LT (ATSWA scheme); the periodic prediction interval T (periodic prediction scheme); and the mean of the underlying exponential distribution l (random prediction scheme).

In some cases, the ROC curve may not be smooth and the superiority of one prediction scheme over the other is difficult to establish. Recent literature describing ROC comparisons includes, for example, Zhang et al., 2002 and Toledano, 2003. Usually, ROC curves are globally summarized by one value, called the area above (or under) the curve. Since the horizontal axis FPR of a seizure warning ROC curve is not bounded, the area above the curve (AAC), given by AAC=∫₀^{∞[}1−f(x)]dx, is the most appropriate measure, where y=f(x), with x and y being the FPR and sensitivity respectively. Smaller AAC indicates better seizure warning performance.

In this seizure warning application, since it is less important to evaluate the performance when sensitivity is low, we have estimated AAC with seizure warning sensitivity at least 50%. For each algorithm, the sensitivity and FPR decreased when the value of its corresponding parameter increased, as expected. For the random prediction scheme, since it essentially is a random process, each point in ROC curve (i.e., for each value of 1) was estimated as the mean sensitivity and mean FPR from 100 Monte Carlo simulations. With 6 different prediction horizons (PHs), inspection of ROC curves suggested that, in comparison with the two naïve seizure warning schemes, ATSWA consistently performed better for lower FPR over almost the entire range of sensitivities. Consistent results were observed from patients with intracranial and scalp EEGs, and from CLE rats.

More specifically, for each patient or rat subject, an AAC was calculated for each of the seizure warning algorithms tested as shown in Table 6. A two-way non-parametric ANOVA test (Friedman's test) was used for overall “algorithm” effects on AAC values. Wilcoxon signed-rank test was then employed to determine the statistical significance of differences of AAC means between any two tested algorithms after an overall significance was observed.

Referring to Table 6, for all patients with intracranial EEG recordings as a whole, when PH=30 minutes, the mean AAC for ATSWA was 0.262. In contrast, the mean areas for the statistical naïve seizure warning schemes were 0.586 (periodic) and 0.666 (random). Friedman's test revealed that there was significant “algorithm” effect (p<0.001) on the observed AAC values. The pairwise comparisons by Wilcoxon sign-rank test showed that the AAC for the ATSWA was significantly less than the AAC from each of the two naïve prediction schemes (p=0.002). Similar results were observed when applying other prediction horizons ranging from 60 to 180 minutes (Table 6A), as well as for patients with scalp EEG recordings (Table 6B) and for epileptic rats (Table 6C).

From the results of this analysis we can conclude that the information extracted from analyses of EEG by ATSWA is statistically significant, and potentially very useful for epileptic seizure warning.

TABLE 6
Comparison of AAc Analysis Using ATSWA and Periodic and
Random Schemes.

| | | | |
|---|---|---|---|
| Prediction | | Periodic | Random |
| Horizon (PH) | ATSWA | Scheme | Scheme |

A. Patients with Intracranial EEG Recordings

| | | | | |
|---|---|---|---|---|
| 30 | minutes | 0.262 | 0.586 | 0.666 |
| 60 | minutes | 0.142 | 0.252 | 0.317 |
| 90 | minutes | 0.105 | 0.168 | 0.201 |
| 120 | minutes | 0.078 | 0.121 | 0.146 |
| 150 | minutes | 0.066 | 0.095 | 0.115 |
| 180 | minutes | 0.053 | 0.079 | 0.095 |

B. Patients with Scalp EEG Recordings

| | | | | |
|---|---|---|---|---|
| 30 | minutes | 0.274 | 0.580 | 0.635 |
| 60 | minutes | 0.140 | 0.270 | 0.301 |
| 90 | minutes | 0.099 | 0.163 | 0.187 |
| 120 | minutes | 0.079 | 0.114 | 0.133 |
| 150 | minutes | 0.059 | 0.087 | 0.102 |
| 180 | minutes | 0.051 | 0.069 | 0.082 |

C. Epileptic Rats with Intracranial EEG Recordings

| | | | | |
|---|---|---|---|---|
| 1 | hours | 0.138 | 0.304 | 0.339 |
| 2 | hours | 0.090 | 0.133 | 0.165 |
| 3 | hours | 0.065 | 0.093 | 0.109 |
| 4 | hours | 0.046 | 0.069 | 0.083 |
| 5 | hours | 0.043 | 0.052 | 0.066 |
| 6 | hours | 0.032 | 0.043 | 0.056 |

### Example 5—Effect of Electrical Stimulation on EEG Morphology and Dynamics During the Interictal State

Animal studies. Adult male Sprague Dawley rats were used for some experiments. The electrical stimulation experiments were conducted in the Children's Miracle Network Animal Neurophysiology Laboratory (ANL) and offline analysis was conducted in the Brain Dynamics Laboratory (BDL) and Computational Neuroengineering Laboratory (CNEL) at the University of Florida. Animals models were developed using modified chronic hippocampal stimulation (CHS) protocol first proposed by Lothman and Bertram (Lothman et al., 1993).

EEG recordings were obtained from four stereotactically implanted electrodes in the bilateral hippocampii and frontal cortical structures. The animals were connected to an automated seizure warning system that ran in parallel with the EEG data acquisition system (STELLATE™ Inc.).

Each animal first underwent a procedure for determining its afterdischarge (AD) threshold. Biphasic square wave pulse trains (AM Systems Inc.) were delivered using bipolar electrodes in the hippocampus, with the two prongs of the electrode acting as the anode and cathode. With the following stimulation parameters constant, (1) frequency=125 Hz, (2) train duration=10 seconds, and (3) pulse width=400 mseconds, the output current intensity was increased from an initial low value in small increments (10˜20 mA) until after discharges (ADs) were observed in the simultaneously recorded EEG.

A stimulation-response study was conducted during the interictal state to study the effects of varying intensity on EEG morphology as well as dynamics. Output current intensities of 50, 75, 100, 125 and 150 mA were used and remained below AD threshold in all experiments. High frequency stimulation was chosen because of reported anticonvulsive effects with hippocampal and amygdalohippocampal stimulation in human subjects with refractory temporal lobe epilepsy (Velasco et al., 2000, Vonck et al. 2002).

A STLmax-based online seizure warning algorithm (ATSWA) ran in parallel with the EEG data acquisition on a separate PC that computed and plotted dynamical and statistical values in real-time. Once the animal was connected to the ATSWA, a training session was used to choose the appropriate electrode combinations to monitor and issue warnings. Upper and lower T-index thresholds were fixed at 5 and 2.662 respectively and a warning was issued when any of the monitored electrode groups showed an entrainment transition (transition from an upper threshold to a lower threshold) and stayed below the lower threshold for 5 minutes.

After a warning was observed, a manual switch was used to switch one of the hippocampal bipolar electrodes (both hippocampii were explored over the course of the experiment) from the recording mode to stimulation mode, to deliver a stimulus train. The following parameter settings were chosen for the initial set of trials: Output current intensity=100 mA; frequency=125 Hz; pulse width=400 mseconds and duration=10 seconds. Offline computation of a nonlinear energy operator (Teager energy) from the EEG was also performed to evaluate changes in signal energy as a result of electrical stimulation.

Results. The following electrodes were used for recording: LF—left frontal, RF—right frontal, LH1 & LH2—left hippocampus and RH1 & RH2—right hippocampus. STLmax values were calculated from each of the above channels. In the example shown in FIG. 6 below, the three groups LF-LH1, RF-LH1 and LF-RF-LH1 were identified as the most critical for warning in the training period and were chosen for monitoring. Stimulations delivered to the lesioned hippocampus (lesioned side during CHS) resulted in resetting the T-index in most cases, and in some instances when delivered to the contralateral hippocampus.

An example of stimulation after a seizure warning is shown in FIG. 13. More particularly,

FIG. 13 shows results using an automated seizure warning system in which plots are refreshed every 10.24 seconds. The top four plots of FIG. 13 correspond to 10.24 seconds of EEG from 4 channels. The middle plots correspond to STLmax values estimated from the 4 channels (the last values correspond to current EEG window). The bottom plots show T-index profiles of all possible electrode combinations (indicated on top left). Each T-index value is calculated from a sliding window of 60 STLmax points, with the last values corresponding to STLmax points within the dashed rectangular window. Vertical red and blue lines indicate ‘seizure warning’ and ‘stimulation’ times respectively. Note the resetting (rise in T-index) after the stimulation.

Stimulating the lesioned hippocampus after observation of a warning produced a rise in the T-index and also seemed to abort, if not prolong, the time to the next seizure. It was observed that the same stimulation parameters were more effective (both in terms of time taken for resetting as well as time to next seizure) when delivered to the lesioned side of the hippocampus rather than the contralateral side. Also, stimulating the lesioned side seemed to give a relatively abrupt increase in the T-index (disentrainment) while stimulation of the contralateral side showed a more gradual disentrainment.

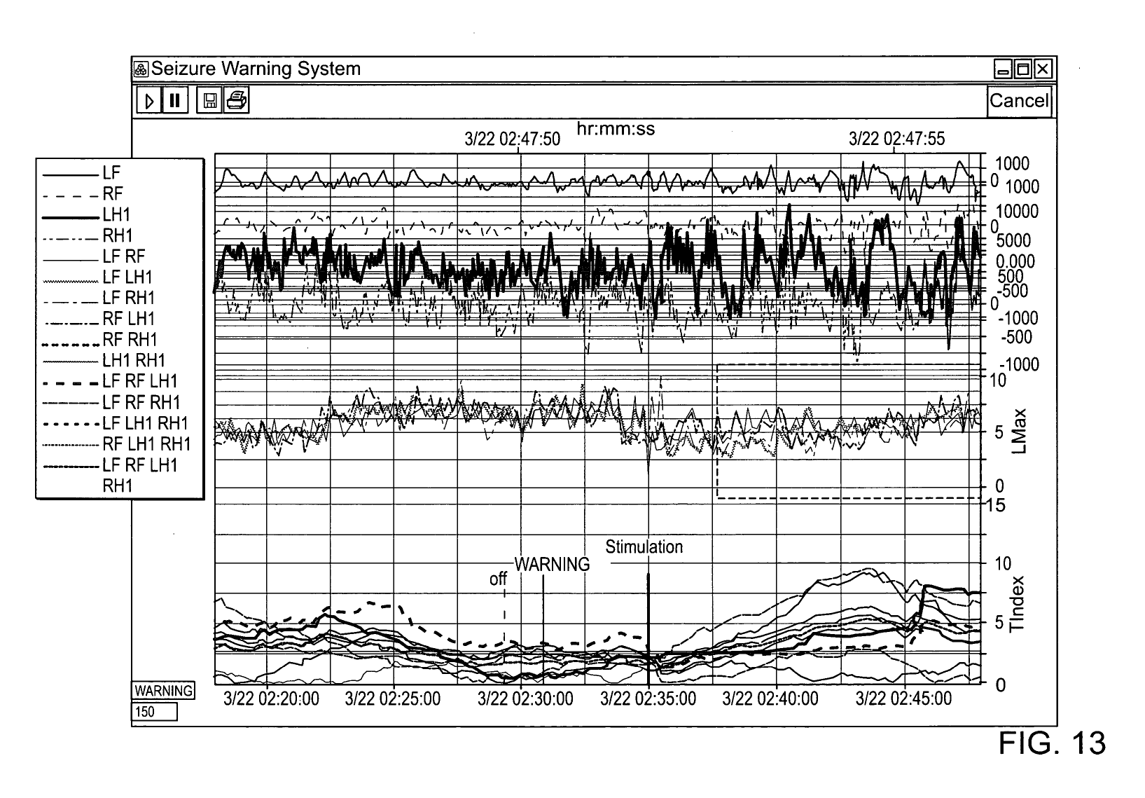

An illustration of EEG changes as a result of hippocampal stimulation is shown in FIG. 14. More particularly, FIG. 14 illustrates post stimulation EEG changes and corresponding dynamical changes in STLmax and T-index. The vertical dashed line indicates the start of stimulation. The top two 30 second EEG segments from four channels illustrate change in EEG morphology before and after a stimulus.

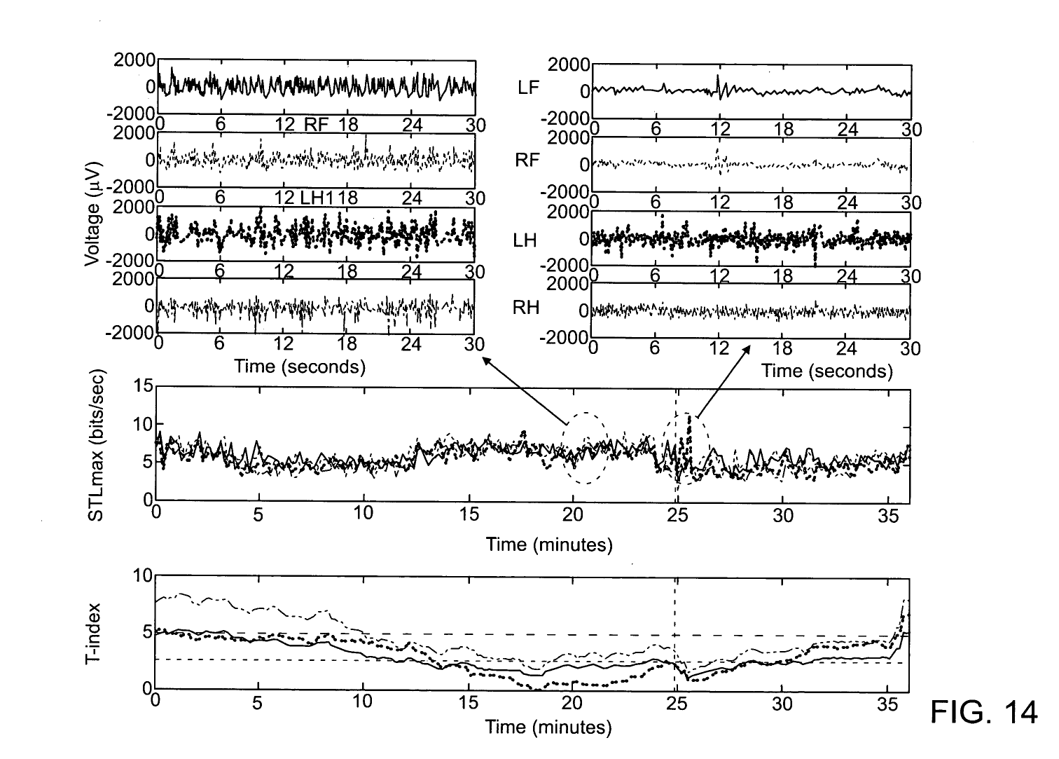

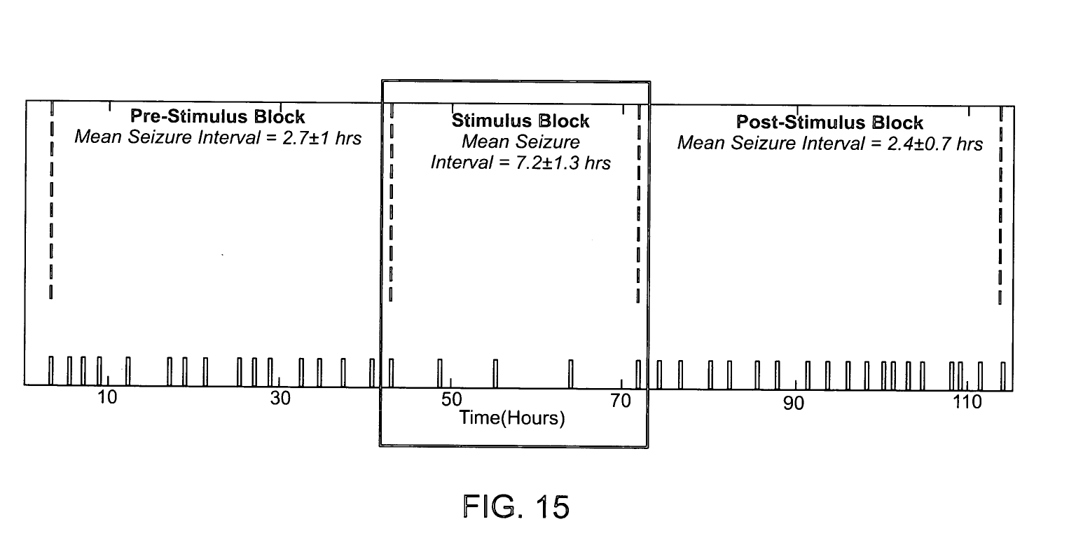

The time after a seizure warning at which a stimulus was delivered seemed to be a significant factor in its ability to reset the brain to the desired interictal (normal) state. We observed that stimulation within 10 minutes of the warning seemed to be more effective than longer wait periods. Warning based stimulation of the lesioned hippocampus also seemed to have an effect on the seizure frequency. Seizure distribution, before and after a stimulus block, is shown in FIG. 15. As can be appreciated, there is significant increase in the inter-seizure interval during the stimulus block, compared to the pre-stimulus and post-stimulus blocks. The increase in inter-seizure interval differed by more than a factor of 2 during the stimulus block compared to the periods when there was no stimulation applied. By contrast, the difference of mean inter-seizure intervals between pre-stimulus and post-stimulus blocks (FIG. 15) was not significant (p>0.5, by Wilcox Rank-Sum test).

Another interesting observation was that resetting was also accompanied by a significant decrease in energy (Teager energy) calculated from the frontal electrodes after the stimulation. Table 7 gives an example of how pre and post stimulation dynamical values were documented and compared.

TABLE 7
Typical Pre and Post stimulus Dynamical and Statistical Values and Time
to Next Seizure

| Experiment | | | | | | | | | | | |
|---|---|---|---|---|---|---|---|---|---|---|---|
| Parameters | | | | Observations | | | | | | | |
| (Intensity; | | | | Mean Teager | | Mean | | T-index | | | |
| Train | | | | Energy | | STLₘₐₓ | | | Time | Duration of | Time to |
| Duration; | | | | Pre- | Post- | Pre- | Post- | | to | Reset | next |
| Frequency; | | | | Stimulus | Stimulus | stimulus | stimulus | Electrode | reset | State | seizure |
| Pulse width) | | Procedure | Location | (10 min) | (10 min) | (10 min) | (10 min) | Group | (min) | (min) | (minutes) |
| 1 | 100 μA; | Stimulus | Left H. | LF: 5.5 | LF: 1.6 | LF: 7.0 | LF: 5.5 | LF-LHI | 10.4 | 24.7 | 240.2 |
| | 10 s; | delivered | | RF: 5.7 | RF: 2.0 | RF: 6.8 | RF: 5.4 | RF-LHI | 9.5 | 5.1 | |
| | 125 Hz; | 7.4 minutes | | LHI: 17 | LHI: 17.5 | LHI: 6.4 | LHI: 4.5 | LF-RF- | 10.7 | 14.4 | |
| | 400 μs | after | | RHI: 9.9 | RHI: 9.3 | RHI: 6.7 | RHI: 4.3 | LHI | | | |
| | | warning | | | | | | | | | |

### Example 6—Embodiments of Closed-Loop Seizure Control Systems

This Example describes several preferred embodiments of seizure control systems in accordance with the present invention.

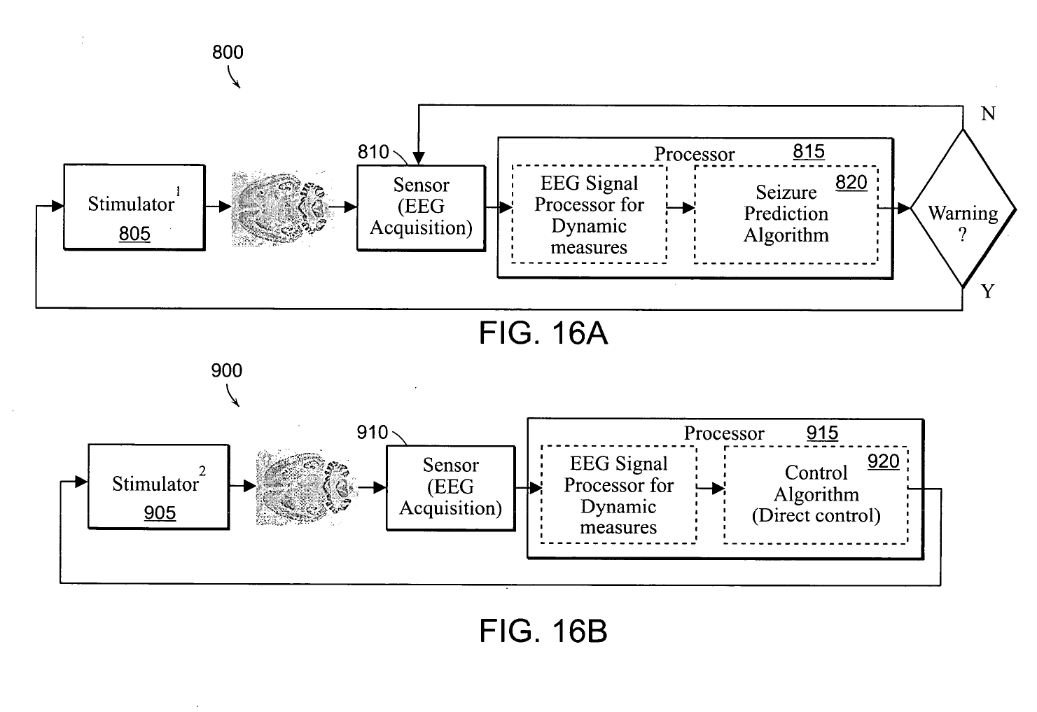

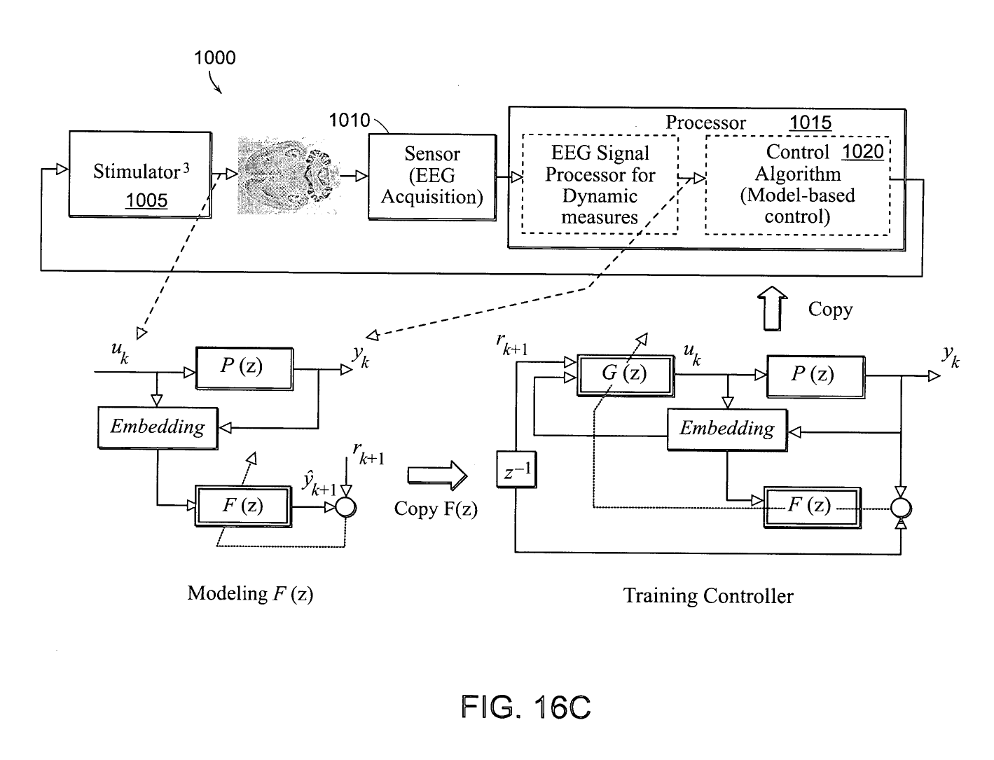

Some of the embodiments are illustrated in FIGS. 16A-C. As discussed above, and shown in FIGS. 16 A, B, and C, the systems each comprise an EEG signal processor (815, 915, and 1015, respectively, in FIGS. 16A-C) for processing dynamic measures. Dynamic descriptors of an EEG to quantify a dynamical state in a neural structure can be selected from the group consisting of STLmax, Similarity index, Kolmogorov entropy, stationarity index, pattern match statistics, recurrence time statistics, and F-statistics.

FIG. 16 A is a block diagram illustrating components of a state-dependent seizure prevention system 800 controlled in accordance with the inventive methods. Within the stimulator 805, control parameters are predetermined and thus the stimulator 805 is kept turned on for a fixed period of time. Seizure prediction algorithm 820 performs a real-time extraction of electrophysiological features associated with a pre-seizure state in the neural structure, as described in further detail in U.S. Pat. No. 6,304,775 and co-pending patent applications U.S. patent application Ser. No. 10/648,354, PCT/US2003/026642, and U.S. patent application Ser. No. 10/673,329, incorporated by reference herein in their entireties. By means of the incorporated seizure prediction algorithm 820, the state-dependent intervention system 800 determines when electrical stimulus intervention is triggered. In this embodiment, only stimulation timing is provided by the system 800 such that the stimulator 805 is turned on for a predetermined duration with fixed stimulation parameters.

FIG. 16B is a block diagram illustrating a direct control system 900 in accordance with the invention. Control parameters of the stimulator 905 are determined by a direct-control algorithm utilizing the state (output from the dynamical descriptors), and the stimulator 905 is kept turned on until a given criterion by a controller is satisfied. More particularly, utilizing a direct control method in which a control law is derived directly from the state of the neural structure (output from the dynamical descriptors), the system 900 checks to determine if there is seizure-associated activity and determines the parameters of the stimulator 905. Similar to a model-based control system (e.g., see further description and FIG. 16C, infra), stimulation parameters such as timing, amount and duration of stimulation are determined by a control law depending only on a change of the system state. Embodiments of the direct control method can be controlled by a delay-feedback or OGY method, as described above.

FIG. 16C provides a schematic diagram of yet a further embodiment 1000, in this case a model-based control system, in accordance with the invention. Control parameters of the stimulator 1005 are determined by a model-based control algorithm. The algorithm is based on a model that represents the relationship between the dynamical descriptor and the stimulator output. In this system, the stimulator is kept turned on until a given criterion by a controller is satisfied. Accordingly, determination of control parameters of the stimulator 1005 is based on a model that quantifies the relationship between the dynamical descriptors and the electrical stimulation output signals. The model can be a global nonlinear model (e.g., recurrent neural networks, time lagged feedforward neural networks) or a multiple switching local linear model. Once the type of model is determined the controller is built in series with the subject, to provide a designated output from the descriptor. In this embodiment not only timing of stimulation, but also the amount and the duration of stimulation are determined by a control law.

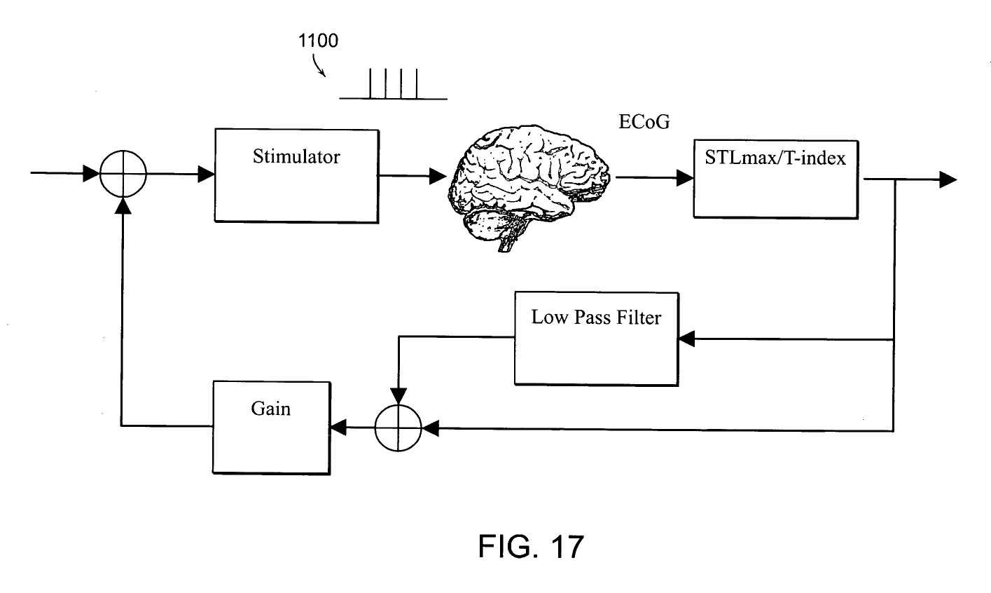

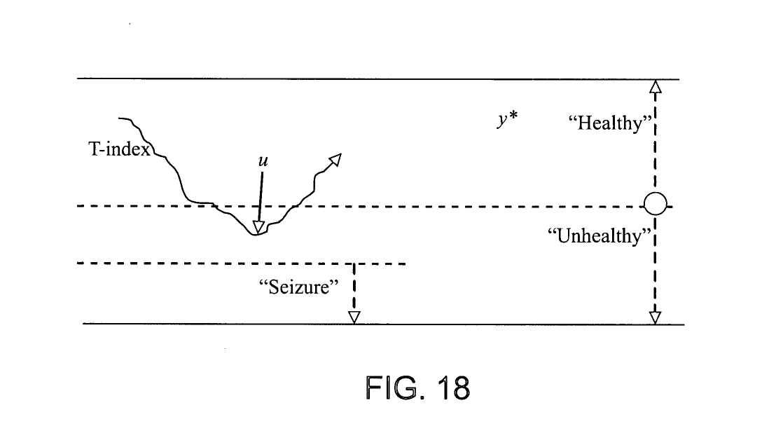

FIGS. 17 and 18 illustrate embodiments of the invention that incorporates methods of direct control. Direct control substitutes the human operator and/or inputs heuristically with a control law derived from the system state. Below are described two embodiments that incorporate methods found to be productive in the control of complex dynamical systems sensitive to initial conditions, i.e., delay feedback control and the OGY method.

Delay Feedback Control (DFC) Method.

Delay Feedback Control is a relatively simple technique applicable to a large class of complex dynamical systems that are sensitive to initial conditions (commonly called chaotic systems but not limited to these) (Pyragas, 1992). The basic idea is to feedback the output of the system to its input, combined with a delayed and processed version of the output. An advantage of this technique for epilepsy seizure applications is that system dynamical equations are not required. A disadvantage is the choice of the operating point to be controlled, and the parameterization needed in the delay. Aspects of embodiments of the system incorporating delay feedback control are illustrated in FIGS. 17 and 18.

Similar to the state-dependent control system, the operating point of the controller is selected based on an ASW algorithm. FIG. 17 schematically illustrates the simplest example of a controller that utilizes a conventional low-pass filter with only one inherent degree of freedom. Once a preictal state is detected by ASW algorithm, the controller is activated to determine the most appropriate stimulation output (parameters). The optimal intensity and frequency is chosen, and the duration of the stimulation is determined automatically by the controller based on the feedback response measure: Dy=yₜ−yₜ*, where yₜ is the T-index value at time t, and yₜ* is the low pass filter value of yₜ. The filtered output yₜ* is used to estimate the location of the fixed point, so that the difference Dy can be used as a feedback perturbation.

Additionally, another condition is added such that the controller is activated to avoid “unhealthy” regions even though Dy=0. The aim here is to construct a reference-free feedback perturbation that automatically locates and stabilizes the T-index values in the fixed-point region, as illustrated in FIG. 18, which shows a desired effect of T-index by a controller. A final step is to find the optimal relationship (i.e., the “gain” in FIG. 17) between Dy and the stimulation duration that gives the best control performance.

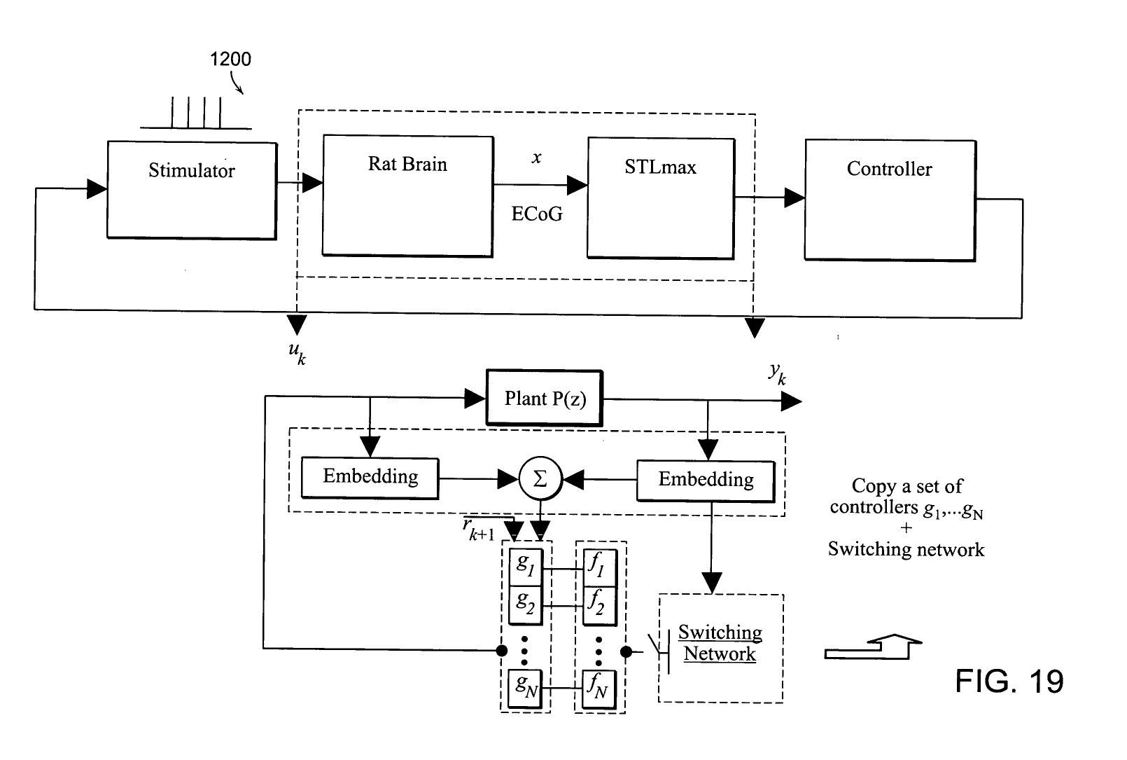

FIG. 19 illustrates an embodiment of the invention that incorporates multiple switching local linear models (MSLLM). Multiple Switching local linear models have the advantage of using a “divide and conquer” strategy to simplify the characterization of complex dynamics by clustering the phase space dynamics in more or less homogenous regions that can be well modeled by a linear model. From the linear model a controller can be easily derived using the inverse control framework. MSLLM is applied to control directly the STLmax (or equivalent). This embodiment utilizes a strategy developed to control Unmanned Aerial Vehicles (UAVs) (Cho et al. 2005). In the seizure control application, four models are used, adapted to the inter-ictal, pre ictal, ictal and post ictal periods, each having different STLmax dynamics. A simple linear model may be able to identify each state efficiently. The switching among models is achieved using a self-organizing map that translates the differences in dynamics. The controller is designed using the inverse controller first that can be derived directly from the linear models. If necessary to improve accuracy of the controller a sliding mode approach as described (Cho et al. 2005) may be implemented. This implementation has been tested in nonlinear systems with success, and accordingly the method is applied directly to the STLmax, without using further simulators.

### Example 8—Spatio-Temporal Dynamical Analysis of EEG Signals

This Example provides details of how to calculate State Descriptor of EEG Dynamics.

STLmax and T-index calculation: The initial step for calculating STLmax is phase space reconstruction. The idea behind this construction is to capture the dynamic of the variables (behavior in time) that are primarily responsible for the global dynamics of the data. In this case, the EEG time series xⱼ(t) from one electrode site j is transformed into a time series of p-dimensional vectors Xⱼ(t)=[xⱼ(t), xⱼ(t+τ) . . . xⱼ(t+(p−1)τ)] using the method of delays with a time lag τ. Theoretically, p should be at least two times the dimension (D) of the formed object in the phase space plus 1 (Takens, 1981).

The measure most often used to estimate D is the phase space correlation dimension ν. Methods for calculating ν from experimental data have been described (Mayer-Kress, 1986; Kostelich, 1992) and were employed in our work to approximate D of the epileptic attractor. In the EEG data we have analyzed, ν was found to be between 2 and 3 during an epileptic seizure. Therefore, in order to capture characteristics of the epileptic attractor, we have used an embedding dimension p of 7 for the reconstruction of the phase space. Herein, a value of τ=20 msec is used for the reconstruction of the phase space (based on the dominant frequency of the epileptic attractor).

After the reconstruction of state vectors, STLmax is defined as the average of local Lyapunov exponents Lᵢⱼ in the state space, that is:

$${STL}_{max} = \frac{1}{N} \cdot {\sum }_{N} {L}_{ij} ,$$

where N is the total number of

the local Lyapunov exponents that are estimated from the evolution of adjacent points (vectors) in the state space, and

$${L}_{ij} = \frac{1}{Δ t} \cdot {log}_{2} \frac{\vert X ( {t}_{i} + Δ t ) - X ( {t}_{j} + Δ t ) \vert}{\vert X ( {t}_{i} ) - X ( {t}_{j} ) \vert} ,$$

where Δt is the evolution time allowed for the vector difference δ₀(xᵢⱼ)=|X(tᵢ)−X(tⱼ)| to evolve to the new difference δ_{κ}(xₖ)=|X(tᵢ+Δt)−X(tⱼ+Δt)|, where Δt=k·dt and dt is the sampling period of u(t). If Δt is given in sec, STLmax is in bits/sec.

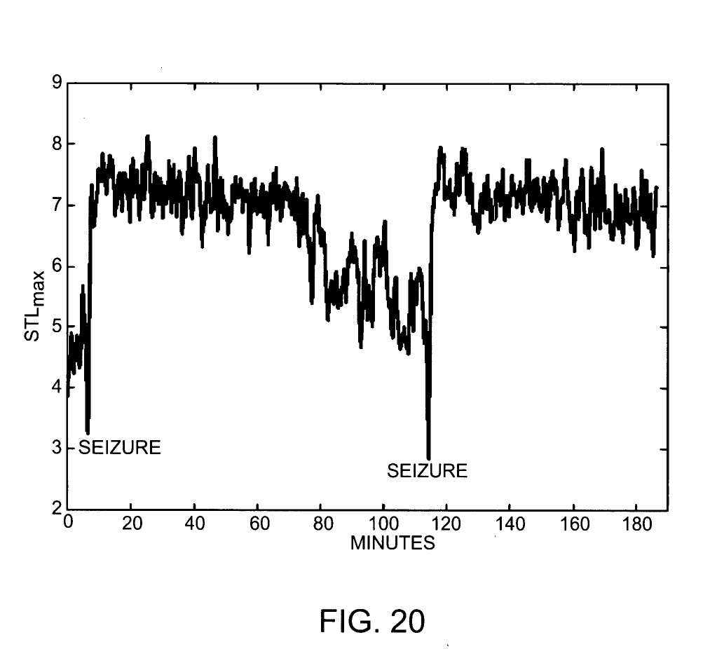

In the STLmax analysis, the EEG time series was divided into non-overlapping segments of 10.24 seconds duration (2048 points). Brief segments were used in an attempt to ensure that the signal within each segment was approximately dynamically stationary. Using the method described in Iasemidis et al. (1990), the STLmax values were calculated continuously over time for the entire EEG recordings. FIG. 20 shows a STLmax profile over approximately three hours including two seizures. More particularly, FIG. 20 shows STLmax profiles over 3 hours including two seizures, and 1 hour after the second seizure. Using embedding dimension p=7 and time delay τ=20 msec for the state space reconstruction, the STLmax values were estimated by dividing the EEG signal into non-overlapping epochs of 10.24 seconds each.

It is seen in FIG. 20 that the values over the entire period are positive. This observation has been a consistent finding in all recordings in all patients studied to date. Moreover, the STLmax values are progressively decreasing from postictal to interictal to preictal periods and reach to the lowest values during the ictal periods. This indicates that methods can be developed, using sequential calculations of STLmax, to detect ictal discharges from the EEG signals.

The main feature in ATSWA is automated detection of dynamical entrainment—defined as gradual convergence of STLmax profiles among critical group of EEG channels. This convergence is quantified by the average T-index based on the pair-T statistic. The T-index for any given pair, calculated over a 10 minute window, is the absolute value of the mean difference in STLmax values divided by the standard deviation. More specifically, the formula for the calculation of a T-index is described below:

For electrode channels i and j, if their STLmax values in a window Wₜ of 60 STLmax points are
Lᵢ^{t}={STL maxᵢ^{t},STL maxᵢⁱ⁺¹, . . . ,STL maxᵢ^{t+59}}
Lⱼ^{t}={STL maxⱼ^{t},STL maxⱼⁱ⁺¹, . . . ,STL maxⱼ¹⁺⁵⁹}
Dᵢⱼ^{t}=Lᵢ^{t}−Lⱼ^{t}={dᵢⱼ^{t},dᵢⱼⁱ⁺¹, . . . dᵢⱼ^{t+59}}
={STL maxᵢ^{t}−STL maxⱼ^{t},STL maxᵢ^{t+1}−STL maxⱼⁱ⁺¹, . . . ,STL maxᵢ^{t+59}−STL maxⱼ^{t+59}},
the pair-T statistic over the time window Wₜ between electrode channels i and j is calculated by

$${T}_{ij}^{t} = \frac{\vert {\overline{D}}_{ij}^{t} \vert}{{\hat{σ}}_{d} / \sqrt{60}} ,$$

where D̅ᵢⱼ^{t} and \hat{σ}_{d} are the average value and the sample standard deviation of Dᵢⱼ^{t}.

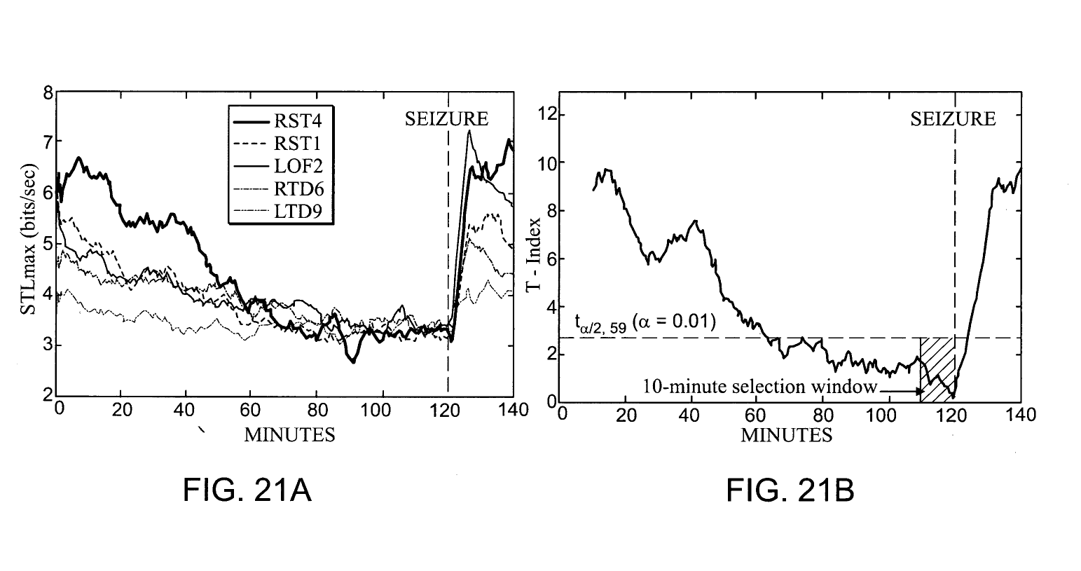

FIG. 21A shows the STLmax profiles over time of 5 electrode sites, selected by the optimization program when it ran in preictal window. FIG. 21B depicts the average T-index value of these sites over time. More specifically, FIG. 21A shows STLmax profiles over 140 minutes, including a 2-minute seizure, for 5 optimally selected electrodes (smoothed by a running moving average within a 1 minute window). FIG. 21B illustrates the average T-index curve and threshold of entrainment from the STLmax profiles in FIG. 21A. The 10-minute preictal window, from which the electrode sites were selected, is also shown in FIG. 21B.

It is noteworthy that the sites selected by the optimization program (RTD6, RST1, RST4, LOF2, LTD9) include the epileptogenic area (RTD, RST), as well as other normal areas (LOF, LTD). This may imply that the spatial extent of the function of the epileptogenic zone in focal epilepsy is much broader than currently believed. It may also be due to variations in the intensity and spatial extent of the physiological effect of the preceding seizure on the phenomenon of resetting that we have investigated (Iasemidis et al., 2004). Moreover, the average T-index of the selected (designated critical) sites over time shows a long trend to lower values as seizure approaches (this observation was the basis for the development of a seizure, prediction algorithm), and is attaining high values rapidly after the seizure. It is also noteworthy that the preictal decline in the T-index values is slower than the postictal rise. This is consistent with the dynamical behavior observed in phase transitions of nonlinear dynamical systems when critical system parameters are moving towards and away from their bifurcation values (Strogatz, 1994). We have called this phenomenon “dynamical resetting of epileptic brain” (Iasemidis et al, 2004), and have used this as a basis and rationale for designing a state-dependent seizure control system.

### REFERENCES

It is believed that a review of the following references will increase appreciation of the present invention. The contents of all patents, patent applications, and references cited throughout this specification are hereby incorporated by reference in their entireties.
Ghai R, Durand D. Effects of applied electric fields on low calcium epilepstiform activity in the CA1 region of rat hippocampal slices. J. Neurophysiol; 26: pp 140-176, 2000.
Warren R, Durand D. Effects of applied currents on spontaneous epileptiform activity induced by low calcium in the rat hippocampus. Brain Res.; 806, pp. 186-195, 1998.
Jerger K, Schiff S J. Periodic pacing of an in vitro epileptic focus. J. Neurophysiol.; 73, pp. 876-79, 1995.
Mirski M A, Rossell L A, Terry J B, Fisher R S. Anticonvulsant effect of anterior thalamic high frequency electrical stimulation in the rat. Epilepsy Res; 28: 89-100, 1997.
Vercueil L, Benazzouz A, Deransart C, et al. High frequency stimulation of the subthalamic nucleus suppresses absence seizures in the rat: comparison with neurotoxic lesions. Epilepsy Res; 31: 39-46, 1998.
Lado F A, Velisek L, Moshe S L. The effect of electrical stimulation of the subthalamic nucleus on seizures is frequency dependent. Epilepsia; 44(2):157-64, 2003.
Fanselow E E, Reid A P, Nicolelis M A. Reduction of pentylenetetrazole-induced seizure activity in awake rats by seizure-triggered trigeminal nerve stimulation. J Neurosci. 1; 20(21):8160-8, 2000.
Oakley J C, Ojemann G A. Effects of chronic stimulation of the caudate nucleus on a preexisting alumina seizure focus. Exp Neurol; 75: 360-67, 1982.
Goodman J H, Berger R E, Tcheng T K. Preemptive Low-frequency Stimulation Decreases the Incidence of Amygdala-kindled Seizures. Epilepsia.; 46(1):1-7, 2005.
Krauss G L, Fisher R S. Cerebellar and thalamic stimulation for epilepsy. Adv. Neurol. 63: 231-45, 1993.
Davis R, Emmonds S E. Cerebellar stimulation for seizure control: 17-year study. Sterotact Funct Neurosurg.; 58: 200-08, 1992.
Velasco F, Velasco M, Ogarrio C, Fanghanel G Electrical stimulation of the centromedian thalamic nucleus in the treatment of convulsive seizures: a preliminary report. Epilepsia, 28: 421-30, 1987.
Velasco F, Velasco M, Jimenez F, et al. Predictors in the treatment of difficult-to-control seizures by electrical stimulation of the centromedian thalamic nucleus. Neurosurgery; 47: 295-304, 2000a.
Velasco M, Velasco F, Velasco A L, Jimenez F, Brito F, Marquez I. Acute and chronic electrical stimulation of the centromedian thalamic nucleus: modulation of reticulo-cortical systems and predictor factors for generalized seizure control. Arch Med Res; 31: 304-15, 2000b.
Velasco F, Velasco M, Jimenez F, Velasco A L, Marquez I. Stimulation of the central median thalamic nucleus for epilepsy. Stereotact Funct Neurosurg; 77: 228-32, 2001.
Hodaie M, Wennberg R A, Dostrovsky J O, Lozano A M. Chronic anterior thalamus stimulation for intractable epilepsy. Epilepsia; 43: 603-08, 2002.
Kerrigan J F, Litt B, Fisher R S, Cranstoun S, French J A, Blum D E, Dichter M, Shetter A, Baltuch G, Jaggi J, Krone S, Brodie M, Rise M, Graves N. Electrical stimulation of the anterior nucleus of the thalamus for the treatment of intractable epilepsy. Epilepsia.; 45(4):346-54, 2004.
Velasco M, Velasco F, Velasco A L, et al. Subacute electrical stimulation of the hippocampus blocks intractable temporal lobe seizures and paroxysmal EEG activities. Epilepsia; 41: 158-69, 2000c.
Vonck K, Boon P, Achten E, De Reuck J, Caemaert J. Long-term amygdalohippocampal stimulation for refractory temporal lobe epilepsy. Ann Neurol; 52: 556-65, 2002.
Osorio I, Frei M G, Sunderam S, Giftakis J, Bhavaraju N C, Schaffher S F, Wilkinson S B. Automated seizure abatement in humans using electrical stimulation. Ann Neurol.; 57(2):258-68, 2005.
Stiver J A and Antsaklis P J. Modeling and analysis of hybrid control systems. Proc. Decision and Control, 4:3748-3751, 1992.
Antsaklis P J. Special issue on hybrid systems: theory and applications a brief introduction to the theory and applications of hybrid systems. Proceedings of the IEEE, 88(7):879-887, 2000.
Bemporad A. Efficient conversion of mixed logical dynamical systems into an equivalent piecewise affine form. IEEE Trans. Automatic Control, 49(5):832-838, 2004.
Bemporad A and Morari M. Optimization-based hybrid control tools. Proc. American Control Conference, 2:1689-1703, 2001.
Koutsoukos X D, Antsaklis P J, Stiver J A and Lemmon M D. Supervisory control of hybrid systems. Proceedings of the IEEE, 88(7):1026-1049, 2000.
Mayer-Kress G, Holzfuss J. Analysis of the human electroencephalogram with methods from nonlinear dynamics. In: Rensing L, ander Heiden U, and Mackey M C, eds. Temporal Disorder in Human Oscillatory Systems. Berlin: Springer Series in Synergetics, Springer-Verlag, 1986; 36:57-68.
Iasemidis L D, Sackellares J C. Chaos theory and epilepsy. The Neuroscientist 1996; 2:118-26.
Iasemidis L D, Sackellares J C, Zaveri H P and Williams W J. Phase space topography of the electrocorticogram and the Lyapunov exponent in partial seizures. Brain Topogr., 2: 187-201, 1990
Iasemidis L D and Sackellares J C. The temporal evolution of the largest Lyapunov exponent on the human epileptic cortex. In: Duck D W and Pritchard W S, eds. Measuring Chaos in the Human Brain. Singapore: World Scientific, 1991, 49-82.
Iasemidis L D, Shiau D S, Sackellares J C, et al. Dynamical resetting of the human brain at epileptic seizures: application of nonlinear dynamics and global optimization techniques. IEEE Transactions on Biomedical Engineering 2004; 51 (3): 493-506.
Pyragus K. Continuous control of chaos by self-controlling feedback. Phys. Lett. A.; 170(6): 421-428, 1992.
Stephanopoulos G Chemical Process Control: An Introduction to Theory and Practice. Prentice-Hall, NJ, 1984.
Bielawski S, Bouazaoui M, Derozier D and Glorieux P. Stabilization and characterization of unstable steady states in a laser. Phys. Rev. A, 47:3276-3279, 1993.
Parmananda P, Rhode M A, Johnson G A, Rollins R W, Dewald H D and Markworth A J. Stabilization of unstable steady states in an electrochemical system using derivative control. Phys. Rev. E, 49:5007, 1994.
Namajunas A, Pyragas K and Tamasevicius A. An electronic analog of the Mackey-Glass system. Physics Letters A, 201:42-46, 1995.
Rulkov N F, Tsimring L S and Abarbanel H D I. Tracking unstable orbits in chaos using dissipative feedback control. Phys. Rev. E, 50:314-324, 1994.
Ciofini M, Labate A, Meucci R. and Galanti M. Stabilization of unstable fixed points in the dynamics of a laser with feedback. Phys. Rev. E, 60:398-402, 1999.
Haken H. Principles of brain functioning: A synergetic approach to brain activity, behavior and cognition, Springer-Verlag, Berlin, 1996.
Ott G, Grebogi C, and Yorke J A. Controlling chaos. Phys. Rev. Lett.; 64(11): 1196-1199, 1990.
Narendra K S and Parthasarathy K. Identification and Control of Dynamical Systems using Neural Networks. IEEE Trans. Neural Networks, 1(1):4-27, 1990.
C. A. Skarda and W. J. Freeman, “How brains make chaos in order to make sense of the world,” Brain Behavioral Science, 10:161-195, 1987.
Freeman W J. Mass Action in the Nervous System. Academic Press, New York, 1975.
Andrievskii B R and Fradkov A L. Control of chaos: Methods and applications. Automation Remote control, 65(4):505-533, 2003.
Hunt E R. Stabilizing High-Period Orbits in a Chaotic System: the Diode Resonator. Phys. Rev. Lett., 67:1953-1955, 1991.
Christini D J, In V, Spano M L, Ditto W L and Collins J J. Real-time experimental control of a system in its chaotic and nonchaotic regimes. Phys. Rev. E, 56:R3749-R3752, 1997.
Slutzky M W, Cvitanovic P and Mogul D J. Manipulating Epileptiform Bursting in the Rat Hippocampus Using Chaos Control and Adaptive Techniques. IEEE Trans. Biomedical Engineering, 50(5):559-570, 2003.
Pyragas K. Continuous control of chaos by self-controlling feedback. Phys. Lett. A, 170:421-428, 1992.
Bleich M E, Hochheiser D, Moloney J V and Socolar J E S. Controlling extended systems with spatially filtered, time-delayed feedback. Phys. Rev. E, 55:2119-2126, 1997.
Loiko N A, Naumenko A V and Turovets S I. Effect of Pyragas feedback on the dynamics of a Q-switched laser. Exper. Theor. Physics, 85:827-834, 1997.
Brandt M E, Shih H T and Chen G R. Linear time-delay feedback control of a pathological rhythm in a cardiac conduction model. Phys. Rev., 56:1334-1337, 1997.
Bleich M and Socolar J E S. Delay feedback control of a paced excitable oscillator. Int. J. Bifurcations and Chaos, 10:603, 2000.
Konishi K, Kokame H and Hirata K. Decentralized delayed-feedback control of a coupled ring map lattice. IEEE Trans. Cir. Syst. 1, 47:1100-1102, 2000.
Jiang G, Chen G and Tang W. Stabilizing unstable equilibria of chaotic systems from a state observer approach. IEEE Trans. Circuits and Systems-II; 51(6): 281-288, 2004.
Hirasawa K, Murata J., Hu J L, et al. Chaos Control on Universal Learning Networks. IEEE Trans. Syst. Man Cybern, 30:95-104, 2000.
Hirasawa K, Wang X F, Murata J, et al. Universal Learning Network and Its Application to Chaos Control. Neural Networks, 13:239-253, 2000.
Poznyak A S, Yu W and Sanchez E N. Identification and Control of Unknown Chaotic Systems via Dynamic Neural Networks. IEEE Trans. Circ. Syst. 1, 46:1491-1495, 1999.
Chen L, Chen G and Lee Y W. Fuzzy Modeling and Adaptive Control of Uncertain Chaotic Systems. Inf. Sci., 121:27-37, 1999.
Tang Y Z, Zhang N Y and Li Y D. Stable Fuzzy Adaptive Control for a Class of Nonlinear Systems. Fuzzy Sets Syst., 104:279-288, 1999.
Dracopoulos D C and Jones A J. Adaptive Neuro-Genetic Control of Chaos Applied to the Attitude Control Problem. Neural Comput. Appl., 6:102-115, 1997.
Weeks E R and Burgess J M. Evolving Artificial Neural Networks to Control Chaotic Systems. Phys. Rev. E, 56:1531-1540, 1997.
Lin C, Liu Y, Chen C and Chen L. Fault accommodation for nonlinear systems using cerebellar model articulation controller. Proc. IJCNN, pp. 879-883, 2004.
Cho J, Lan J, Principe J C and Motter M A. Modeling and Control of Unknown Chaotic Systems via Multiple Models. Proc. MLSP, pp. 53-62, 2004.
Kohonen T, Self-Organizing Maps. Springer-Verlag: New York, 1995.
Haykin S and Principe J C. Dynamic modeling of chaotic time series with neural networks. IEEE Trans. Signal Processing Mag., pp. 66-81, 1998.
Werbos P J. Backpropagation through time: What it does and how to do it. Proceedings of the IEEE. 78(10):1550-1560, 1990.
Elman J L. Finding structure in time. Cognitive Science 14(2): 179-211, 1990.
Siegelmann H T. Foundations of Recurrent Neural Networks. PhD thesis, Rutgers University, New Brunswick, 1993.
Jaeger H. The echo state approach to analyzing and training recurrent neural networks. Technical Report GMD Report 148, German National Research Center for Information Technology, 2001.
Rao Y, Kim S, Sanchez J, Erdogmus D, Principe J C, Carmena J, Lebedev M and Nicolelis M. Learning Mappings in Brain Machine Interfaces with Echo State Networks. to appear in Proc. ICASSP, 2005.
Haykin S, Neural networks: A comprehensive foundation. New York: Macmillan College Publishing Company, 1999.
Narendra K S and Parthasarathy K. Identification and control of dynamical systems using neural networks. IEEE Trans. Neural Networks; 1(1): 4-27, 1990.
Cho J, Principe J C, Erdogmus D and Motter M A. Modeling and Inverse Controller Design for an Unmanned Aerial Vehicle Based on the Self-Organizing Map. to appear in IEEE Trans. Neural Networks, 2006.
Rabiner L R. A tutorial on hidden Markov models and selected applications in speech recognition. Proceedings of IEEE, 77(2):257-286, 1989.
Huang R S, Kuo C J, Tsai L L and Chen O T C. EEG pattern recognition—arousal states detection and classification. Proc. ICNN, 2:641-646, 1996.
Obermaier B, Guger C and Pfurtscheller G. HMM used for the offline classification of EEG data. Biomedizinsche Technik, 1999.
Penny W and Roberts S. Gaussian observation hidden markov models for EEG analysis. Tech. Rep. TR-98-12, Imperial College, London, 1998.
Abarbanel H D I. Analysis of observed chaotic data, New York: Springer-Verlag, 1996.
Abraham N B, Albano A M, Das B, De Guzman G, Yong S, Gioggia R S, Puccioni G P and Tredicce J R. Calculating the dimension of attractors from small data sets. Phys. Lett. A 114: 217, 1986.
Liebert W and Schuster H G Proper choice of the time delay for the analysis of chaotic time series. Phys. Lett. A 142: 107, 1989.
Rosenstein M T, Collins J J and De Luca C J. A practical method for calculating largest Lyapunov exponents from small data sets. Physica D 65:117-134, 1993.
Kruel T M, Eisworth M and Schreider F M. Introduction to non-linear mechanics. Princeton University Press, Princeton, N.J., 1993.
Kantz H. A robust method to estimate the maximum Lyapunov exponent of a time series. Physics Letters A 185: 77-87, 1994.
Eckmann J P and Ruelle D. Ergodic theory of chaos and strange attractors. Reviews of Modern Physics 57: 617-656, 1985.
Sano M and Sawada Y: Physical Review Letters 55: 1082-1085, 1985.
Sauer T D, Tempkin J A and Yorke J A. Spurious Lyapunov exponents in attractor reconstruction. Physical Review Letters 81: 4341-4344, 1998.
Sauer T D and Yorke J A. Reconstructing the Jacobian from data with observational noise. Physical Review Letters 83: 1331-1334, 1999.
Pardalos P M, Yatsenko V A and Cifarelli C. On the computation of lyapunov exponents using optimization. Submitted for print to Journal of Optimization methods and software 2005.
Mosher J C and Leahy R M. Source localization using recursively applied and projected (RAP) MUSIC IEEE Trans. Signal Process. 47: 332-40, 1999
Xu X-L, Xu B and He B. An alternative subspace approach to EEG dipole source localization. Phys. Med. Biol. 49: 327-343, 2004.
Kolmogorov A N. The general theory of dynamical systems and classical mechanics, In: Foundations of Mechanics, Abraham R. and Marsden J. E. (Eds.), 1954.
Palus M, Albrecht V and Dvorak I. Information theoretic test for nonlinearity in time series, Phys. Lett. A. 175: 203-209, 1993.
Elgammal A, Duraiswami R and Davis L S. Efficient Kernel Density Estimation Using the Fast Gauss Transform with Applications to Color Modeling and Tracking. Submitted to IEEE Transaction on Pattern Analysis and Machine Intelligence.
Shiau D S. Signal Identification and Forecasting in Nonstationary Time Series Data. Ph.D. Dissertation, University of Florida, 2001.
Shiau D S, Iasemidis L D, Yang M C K, Carney P R, Pardalos P M, Suharitdamrong W, Nair S P and Sackellares J C. Pattern-match regularity statistic—A measure quantifying the characteristics of epileptic seizures. Epilepsia 45 (S7): 85-86, 2004.
Gao J B. Recurrence time statistics for chaotic systems and their application. Physical Review Letters 83(16), 1999.
Takens F. Detecting Strange Attractors in Turbulence. Dynamical Systems and Turbulence Lecture Notes in Mathematics 898, Rand D A and Young L S (Eds.). Springer Verlag, Berlin, 336, 1981.
Hively L M, Protopopescu V A and Gailey P C. Timely Detection of Dynamical Change in Scalp EEG Signal. Chaos 10(4), 2000.
Lai Y C, Osorio I, Harrison M A F and Frei M G Correlation-dimension and Autocorrelation Fluctuation in Epileptic Seizure Dynamics. Physical Review E 65: 031921, 2002.
Iasemidis L D, Principe J C and Sackellares J C. Measurement and Quantification of Spatio-Temporal Dynamics of Human Epileptic Seizures. Nonlinear Signal Processing in Medicine, Akay M (Ed.), IEEE Press 1999.
Yang M C K and Carter R L. One-way analysis of variance with time-series data. Biometrics 39(3), 747-751, 1983.
Pompe B. Measuring statistical dependencies in a time series. Journal of Statistical Physics 73: 587-610, 1993.
Hoyer D, Hoyer O, Zwiener U. A new approach to uncover dynamic phase coordination and synchronization. IEEE transactions on biomedical engineering 47: 68-74, 2000.
Rosenblum M G and Kurths J. Analysing synchronization phenomena from bivariate data by means of the Hilbert transform. Nonlinear Analysis of Physiological Data, (Kantz H, Kurths J and Mayer-Kress G, Eds.). 91-99, Springer, Berlin, 1998.
Lachaux J P, Rodriguez E, Martinerie J and Varela F. Measuring phase-synchrony in brain signals. Human Brain Map. 8: 194-208, 1999.
Arnhold J, Grassberger P, Lehnertz K and Elger C E. A robust method for detecting interdependencies: Application to intracranially recorded EEG Physica D 134: 419-430, 1999.
Haykin S. Neural Networks: A Comprehensive Foundation, 2nd edition, Prentice Hall, 1999.
Principe J C, Euliano N R and Lefebvre W C. Neural and Adaptive Systems: Fundamentals Through Simulations, John Wiley & Sons, 2000.
Hegde A, Erdogmus D, Rao Y N, Principe J C, Gao J: “SOM-Based Similarity Index Measure: Quantifying Interactions Between Multivariate Structures,” Proceedings of NNSP'03: 819-828, Toulouse, France, September 2003.
Pardey J, Roberts S and Tarassenko L. A review of parametric modelling techniques for EEG analysis, Medical Engineering & Physics 18(1): 2-11, 1996.
Rezek I A and Roberts S J. Stochastic complexity measures for physiological signal analysis, Biomedical Engineering, IEEE Transactions 45(9):1186-1191, 1998.
Christian E E and Lehnertz K. Can epileptic seizures be predicted evidence from nonlinear time series analysis of brain electrical activity? Phys. Rev. Lett. 80(22):5019-5022, 1998.
Lewicki M S. A review of methods for spike sorting: The detection and classification of neural action potentials, Network: Computation Neural Syst. 9: R53-R78, 1998.
Pierre C. Independent component analysis. A new concept? Signal Processing 36(3): 287-314, 1994.
Han J and Kamber M. Data Mining: concepts and techniques, Academic press, 2001.
Duda R O, Hart P E and Stork D G Pattern classification, Wiley-Interscience, New York, 2001.
Vapnik V N. The nature of statistical learning theory. Springer Verlag, New York, 1995.
Burges C J C. A tutorial on support vector machines for pattern recognition, Data Mining and Knowledge discovery 2(2):121-167, 1998.
Paxinos Q Watson C. The Rat Brain in Stereotaxic Coordinates, Fourth Edition. New York: Academic Press, 1998.
Feng P, Vogel G W. A new method for continuous, long-term polysomnographic recording of neonatal rats. Sleep; 23(8):1005-14, 2000.
Carney P R, Nair S P, Iasemidis L D, Shiau D S, Pardalos P M, Shenk D, Norman W M, and Sackellares J C. Quantitative analysis of EEG in the rat limbic epilepsy model. Neurology 62 (7, Suppl. 5), A282-A283, 2004.
Nair S P, Shiau D S, Norman W M, Shenk D; Suharitdamrong W, Iasemidis L D, Pardalos P M, Sackellares J C, Carney P R Dynamical changes in the rat chronic limbic epilepsy model. Epilepsia (Suppl), 2005.
Vere-Jones, 1995. Inventors please note this reference is not on Grant list.
Litt, B., Esteller, R., Echauz, J., D'Alessandro, M., Short, R., Henry, T., Pennell, P., Epstein, C., Bakay, R., Dichter, M., Vachtsevanos, G, Epileptic seizures may begin hours in advance of clinical onset: a report of five patients. Neuron 30, 51-64, 2001.
Andrzejak R G, Kreuz T, Mormann F, Kraskov A, Rieke C, Elger C E, Lehnertz K: Put your seizure prediction statistics to the test: The method of Seizure Time Surrogates. Epilepsia; 44(9): 172, 2003.
Andrzejak R G, Mormann F, Kreuz T, Rieke C, Kraskov A, Elger C E, Lehnertz K: Testing the null hypothesis of the non-existence of a pre-seizure state. Phys Rev E; 67, 010901, 2003.
Zhang D D, Zhou X H, Freeman D H, Freeman J L. A non-parametric method for comparison of partial areas under ROC curves and its application to large health care data sets. Statistics in Medicine 2002; 21:701-715.
Toledano A Y. Three methods for analyzing correlated ROC curves: a comparison in real data sets from multi-reader, multi-case studies with a factorial design. Statistics in Medicine 2003; 22:2919-2933.
Lothman E W, Bertram E H, Bekenstein J W, Perlin J B. Self-sustaining limbic status epilepticus induced by ‘continuous’ hippocampal stimulation: electrographic and behavioral characteristics. Epilepsy Res.; 3(2): 107-19, 1989.
Lothman E W, Bertram Eh, Kapur J, Stringer J L. Recurrent spontaneous hippocampal seizures in the rat as a chronic sequela to limbic status epilepticus. Epilepsy Res; 6(2): 110-8, 1990.
Lothman E W, Bertram E H 3^{rd}, Stringer J L. Functional anatomy of hippocampal seizures. Prog Neurobiol.; 37(1): 1-82, 1991.
Velasco M, Velasco F, Velasco A L, et al. Subacute electrical stimulation of the hippocampus blocks intractable temporal lobe seizures and paroxysmal EEG activities. Epilepsia; 41: 158-69, 2000.
Vonck K, Boon P, Achten E, De Reuck J, Caemaert J. Long-term amygdalohippocampal stimulation for refractory temporal lobe epilepsy. Ann Neurol.; 52: 556-565, 2002.
Yang M C K and Carter R L. One-way analysis of variance with time-series data. Biometrics 39(3), 747-751, 1983.

The invention has been described in detail with reference to preferred embodiments thereof. However, it will be appreciated that those skilled in the art, upon consideration of this disclosure, may make modifications and improvements within the spirit and scope of the invention.

What is claimed is:

1. A closed-loop state-dependent neuroprosthetic device for seizure prevention wherein control of electrical stimulation intervention is determined by the dynamical electrophysiological state of a neural structure being monitored, comprising:
a detection system that detects and collects electrophysiological information detectable by electroencephalography (EEG) from a neural structure in a subject;
an analysis system that evaluates the detected and collected electrophysiological information and performs a real-time extraction of said information to obtain electrophysiological features associated with a pre-seizure state in the neural structure, and from the extracted features determines when electrical stimulus intervention is required; and
an electrical stimulation intervention system that provides electrical stimulation output signals having desired stimulation parameters to a neural structure being monitored and in which abnormal neuronal activity is detected;
wherein the analysis system further analyzes collected electrophysiological information following electrical stimulation intervention to assess the short-term effects of the stimulation intervention and to provide feedback to maintain or modify such stimulation intervention.

2. The closed-loop state-dependent neuroprosthetic device of claim 1, further comprising an electrode array being configured to selectively detect electrophysiological information detectable by electroencephalography (EEG), and to output the electrical stimulation output signals;
wherein the electrode array is configured so as to create a plurality of channels and wherein said providing electrical stimulation output signals includes providing electrical stimulation output signals having desired stimulation parameters to one or more of the plurality of channels, in which in said one or more channels it is predicted or determined that there is the onset of an epileptic state.

3. The closed-loop state-dependent neuroprosthetic device of claim 1, further comprising an algorithm for automated seizure warning (ASWA).

4. The closed-loop state-dependent neuroprosthetic device of claim 1, wherein the ASWA comprises algorithms for dynamical analysis of EEG signals, for selection of electrode groups and for statistical pattern recognition detecting a seizure-associated state.

5. A method for preventing or delaying a seizure, comprising the steps of:
monitoring electroencephalographic (EEG) recording signals in at least one neural structure in a subject fitted with a closed-loop state-dependent neuroprosthetic device for seizure prevention wherein control is determined by the dynamical electrophysiological state of a neural structure subject to seizure;
detecting and collecting electrophysiological information obtained from the neural structure;
analyzing the detected and collected electrophysiological information;
performing a real-time extraction of said information to obtain electrophysiological features associated with a pre-seizure state in a neural structure being monitored;
predicting from the real-time extraction of said features the onset of an epileptic state in said neural structure; and
providing electrical stimulation intervention output signals having desired stimulation parameters to at least a portion of a neural structure predicted to assume an epileptic state, sufficient to prevent or delay the occurrence of a seizure in the neural structure.

6. The method of claim 5, further comprising the steps of:
providing an electrode array being configured to selectively detect electrophysiological information detectable by electroencephalography, and to output the electrical stimulation output signals, wherein the electrode array is configured so as to create a plurality of channels and wherein said providing electrical stimulation output signals includes providing electrical stimulation output signals having desired stimulation parameters to one or more of the plurality of channels, in which in said one or more channels it is predicted or determined that there is the onset of an epileptic state.

7. The method of claim 5, further comprising the steps of:
collecting electrophysiological information during or following said providing stimulation output signals;
analyzing the collected information and assessing the short-term effects of the stimulation output signals on the onset of the epileptic state;
determining if there is one of increased, decreased or maintenance of seizure-associated activity from said analyzing; and
maintaining or modifying the stimulation output signals being provided, based on the determined increase, decrease or maintenance of seizure-associated activity.

8. The method of claim 5, wherein the neural structure being recorded is within a region of the brain selected from the group consisting of the limbic system, hippocampus, entorhinal cortex, CA1, CA2, CA3, dentate gyrus, hippocampal commissure, thalamic nuclei (e.g., anterior and centromedian), subthalamic nucleus, and other basal ganglia.

9. The method of claim 5, wherein determination of parameters of the electrical stimulation intervention output signals is based on a direct control method in which a control law is derived from the state of the neural structure.

10. The method of claim 9, wherein the direct control method comprises a delay feedback control method.

11. The method of claim 9, wherein the direct control method comprises an Ott, Grebogy and York (OGY) method.

12. The method of claim 5, wherein determination of parameters of the electrical stimulation intervention output signals is based on a model that utilizes macroscopic modeling of the dynamical descriptors of brain electrical activities.

13. The method of claim 12, wherein the model quantifies the relationship between the dynamical descriptors and the electrical stimulation intervention output signals.

14. The method of claim 12, wherein the model comprises a step of determining signal dynamics in an electroencephalogram (EEG) over a segment of time.

15. The method of claim 14, utilizing a Short-Term Maximum Lyapunov (STLmax), exponent-based methodology, or a variation thereof, to quantify a dynamical state in a neural structure.

16. The method of claim 14, utilizing a dynamical descriptor of an EEG selected from the group consisting of Kolmogorov entropy, stationarity index, pattern match statistics, and recurrence time statistics, to quantify a dynamical state in a neural structure.

17. The method of claim 12, wherein the model comprises a hybrid continuous-discrete control scheme.

18. The method of claim 12, utilizing global nonlinear dynamic modeling.

19. The method of claim 12, utilizing multiple switching local linear modeling.

20. The method of claim 12, wherein the local dynamical state is determined for each recording channel on an EEG.

21. The method of claim 12, wherein interdependency between EEG signals (among EEG signal groups) is estimated using a T-index (F-index).

22. The method of claim 12, wherein interdependency between EEG signals is directly estimated from a pair or a group of EEG signals.

23. The method of claim 22, wherein the interdependency measure between signals is estimated using a self-organizing map-based similarity index (SOM-SI).
